(19) United States
(12) Patent Application Publication
(10) Pub. No.: US 2021/0008220 A1
(43) Pub. Date: Jan. 14, 2021

(54) PHARMACEUTICAL COMPOSITIONS CONTAINING POLYROTAXANES

(71) Applicant: UNIVERSITY OF GEORGIA RESEARCH FOUNDATION, INC., Athens, GA (US)

(72) Inventors: May XIONG, Athens, GA (US); Zhi LIU, Athens, GA (US)

(21) Appl. No.: 17/041,215

(22) PCT Filed: Mar. 26, 2019
(86) PCT No.: PCT/US2019/024009

§ 371 Date: Sep. 24, 2020

Related U.S. Application Data
(60) Provisional application No. 62/648,134, filed on Mar. 26, 2018.

(51) Int. Cl. A61K 47/61; C08G 83/00; C08B 37/16; A61K 31/164; A61K 31/4196; A61K 31/4412

(52) U.S. Cl. CPC A61K 47/61; C08G 83/007; A61K 31/4412; A61K 31/164; A61K 31/4196; C08B 37/0015

(57) ABSTRACT

Disclosed herein are a polyrotaxane conjugated metal chelators and selectively cleavable linkers. The compositions have prolonged plasma residence time, and upon contact with the appropriate chemical environment, are cleaved and then renally and fecally cleared.

### CROSS-REFERENCE TO RELATED APPLICATION

This application claims the benefit of U.S. Provisional Application 62/648,134, filed Mar. 26, 2018, the contents of which are hereby incorporated in its entirety.

### STATEMENT ACKNOWLEDGING GOVERNMENT SUPPORT

This invention was made with government support under Grant No. R01DK099596 awarded by the National Institutes of Health. The government has certain rights in the invention.

### FIELD

The invention is directed to polyrotaxane conjugated with pharmaceutical agents. The polyrotaxanes have prolonged plasma residence times, and can be used to improve the pharmacokinetic properties of the pharmaceutical agent.

### BACKGROUND

Iron overload (IO), also known as hemochromatosis, is a condition characterized by excessive iron deposition in critical organs of the body, especially liver and heart. It can arise as a result of genetic conditions or be acquired due to repeated blood transfusions. Individuals with the condition typically exhibit few symptoms in the early stages and are often unaware of their condition until it has already progressed to a dangerous level. IO can induce cirrhosis of the liver leading to an increased risk of developing liver cancer, contribute to the development of arthritis, and can cause impotence in males. Risk factors for IO may also increase even further in patients with diabetes due to selective iron deposition into pancreatic islet β cells which can lead to functional failure of the pancreas. Ultimately, iron accumulation can cause IO-related cardiomyopathies such as abnormal heart rhythms or heart failure, and is the primary cause of mortality in patients. IO is also relevant to neurological diseases, with recent studies demonstrating presence of excess iron in the brains of Alzheimer and Parkinson's disease patients.

The mainstay approach to alleviate IO is to infuse patients for days with the clinically-approved small molecule metal chelator Deferoxamine (DFO). DFO has high binding affinity to ferric iron and is rapidly eliminated from the body. A large body of clinical evidence has demonstrated that survival of patients with IO can be significantly increased through the long-term subcutaneous administration of DFO. In spite of the wide-spread use of this drug, critical drawbacks to DFO therapy involve its non-specific toxicity in humans at high doses when infused intravenously or intramuscularly into patients and short blood circulation times (half-life of ca. 20 min in humans).

There remains a need for improved methods of treating iron overload. There remains a need for improved therapeutic agents for reducing iron plasma concentrations. There remains a need for improved formulations of DFO and other iron chelators with prolonged circulation times and reduced toxicity.

### SUMMARY

Disclosed herein are polyrotaxanes conjugated to metal chelating compounds. The conjugates are sufficiently large to avoid renal clearance, and therefore have prolonged plasma residence times and improved metal sequestering ability. The polyrotaxanes are engineered to degrade in a specified chemical environment, at which time the smaller components are excreted.

The details of one or more embodiments are set forth in the descriptions below. Other features, objects, and advantages will be apparent from the description and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. (A) Schematic representation for preparing rPR-DFO. (B) ¹H NMR spectra of PPR (DMSO-d₆), rPR (DMSO-d₆) and rPR-DFO (D₂O). (C) Representative UV-Vis absorption spectrum of DFO and rPR-DFO in the absence and presence of iron(III). (D) Representative TEM image of rPR-DFO revealed crescent-shaped structures that appeared spherical (scale bar equals 50 nm).

FIG. 2. (A) Degradation of rPR-DFO was monitored by DLS and (B) aqueous GPC before (blue) and after (red) exposure to 100 mM H₂O₂ at 37° C. for 24 h. (C) 2D DOSY ¹H NMR experiments (D₂O) with rPR-DFO demonstrate drastic changes in the diffusion behavior of species in solution before and after exposure to H₂O₂, and conventional ¹H NMR (D₂O) clearly reveals the peak corresponding to the thioketal methyl protons of rPR-DFO disappearing after cleavage, along with the appearance of a new peak corresponding to acetone. The degradation of rPR-DFO is initiated by the de-thioacetalization of thioketal linkages in the presence of ROS, releasing the adamantine endcaps, generating acetone as a by-product, and subsequently resulting in CDs threading off the PEG chains. (D) In vitro degradation of rPR-DFO was further assessed by incubating fluorescent Fc-rPR-DFO with IO macrophage cells (50 and 100 μM FAC). To assess degradation over time, cell culture media was collected at 24 and 48 h timepoints and fluorescence was measured.

FIG. 3. (A) Cytotoxicity of free DFO, rPR-OH and rPR-DFO in NIO and IO macrophage cells after 48 h incubation. (B) Hemolysis of RBCs incubated with rPR-OH and rPR-DFO. Inset: corresponding images of RBCs incubated with rPR-OH and rPR-DFO (20 mg/mL) for 12 h. (C) Ferritin expression levels in IO macrophage cells treated with free DFO, rPR-OH and rPR-DFO; results are normalized to total protein (ng/μg). (D) Time-dependent iron-mediated oxidative stress levels in IO macrophage cells treated with DFO, rPR-OH, and rPR-DFO. (E) IO and NIO macrophage cells were incubated with Fc-rPR-DFO for 4 h prior to adding LysoTracker and imaging with a confocal microscope (scale bar: 20 μm). Where relevant, all the data is presented as mean±SD (n=3); “ns” means the difference was not significant and ***p<0.001.

FIG. 4. (A) Biodistribution of NIR-labeled rPR-DFO (SCy5.5-rPR-DFO) was visually monitored in NIO and IO mice up to 192 h. (B) Ex vivo fluorescence images of (left to right) liver, spleen, heart, kidneys, and lungs harvested from NIO and IO mice at 24 h and 192 h post-injection of SCy5.5-rPR-DFO. (C) Quantitative biodistribution of SCy5.5-rPR-DFO in various organs for NIO and IO mice at 4, 12, 24 and 196 h. Results are presented as mean±SD (n=6). (D) Pharmacokinetic profiles and parameters for Scy5.5-rPR-DFO in NIO and IO mice. (E) Ex vivo fluorescence images of a sample of feces and urine collected from NIO mice treated with saline and SCy5.5-rPR-DFO, and IO mice treated with SCy5.5-rPR-DFO.

FIG. 5. (A) Bw and (B) ow of NIO mice 14 days after a 6-dose (100 mg/kg/dose eq. DFO) treatment regimen. (C) Representative H&E-stained photomicrographs of liver, spleen, heart, kidney, lung, and brain of NIO mice administered 6 doses of saline, DFO, rPR-OH, or rPR-DFO. (D) Liver function was monitored by measuring ALT, ALP and AST and kidney function was monitored by measuring BUN and CRE. Statistics comparing the different treatment groups are summarized in the table, where results are presented as mean±SD (n=6) and “ns” means the difference was not significant.

FIG. 6. (A) Serum ferritin levels in IO mice treated with 3 (red) or 6 (blue) doses of either saline, DFO or rPR-DFO at 100 mg/kg/dose eq. DFO or eq. rPR-OH. The results are presented as mean±SD (n=6); “ns” means the difference was not significant, ***p<0.001, and ****p<0.0001. (B) Total iron elimination levels in urine and feces of IO mice after 6 doses; the results are presented as mean±SD (n=6) where “ns” means the difference was not significant, *p<0.05, and ****p<0.0001.

FIG. 7. (A) Experimental timeline for assessing the LIL in IO mice using MR-based R₂* quantification. Representative dynamic R₂* distribution histograms are shown (left), as well as the corresponding gradient echo (GE) magnitude (right top) and representative R₂* images (right bottom) are shown for the same mouse at each time point: (row B) untreated NIO, (row C) untreated IO, (row D) IO treated with DFO, (row E) IO treated with rPR-OH, and (row F) IO treated with rPR-DFO.

FIG. 8A depicts a synthetic scheme for preparing TEC. FIG. 8B depicts a synthetic scheme for obtaining polyrotaxanes.

FIG. 9 (A) Stability of rPR-DFO at pH 5, 7.4, and 10 was monitored by DLS at 24 and 240 h. (B) Representative UV-Vis absorption spectrum of the top concentrate and bottom filtrate of rPR-DFO:iron(III) before and after exposure to 100 mM H₂O₂ at 37° C. for 24 h. (C) Time and iron concentration increase iron-mediated oxidative stress levels in cells; ROS levels were measured through the ROS-sensitive fluorescent probe DCFDA.

FIG. 10 Cytotoxicity of (A) the degraded products of rPR-DFO and rPR-OH and (B) acetone (a byproduct of thioketal cleavage) in NIO and IO macrophage cells after 48 h incubation; a representative set of data is shown where each data point is presented as the mean±SD (n=3).

FIG. 11 The MTD of rPR-DFO was investigated in NIO mice receiving single escalating doses of rPR-DFO at eq. DFO concentrations. Acute toxicity in animals was assessed by measuring (A) BW of mice every other day up to 14 days post-injection and (B) OW at the end of the study; OW was normalized with respect to the total weight of each animal. (C) Representative histological photomicrographs of organ sections stained with H&E following 500 mg/kg of rPR-DFO or eq. rPR-OH treatment (20× magnification, scale bar=50 μm).

FIG. 12 BW of IO mice administered (A) 3 doses and (B) 6 doses of saline, DFO or eq. rPR-DFO, or eq. rPR-OH. OW in IO mice administered (C) 3 doses and (D) 6 doses of saline, DFO or eq. rPR-DFO, or eq. rPR-OH. Results are presented as mean±SD (n=3). “ns” means the difference was not significant.

FIG. 13 Averages and standard deviations of liver R₂* measurements for each mouse at time points indicated. Day 8, 15, and 22 measurements were determined to be significantly different from Day 1 measurements based on whether the absolute value of Glass' Δ was greater than 0.8 (measurements denoted by *, see Table S1).

FIG. 14 Representative photomicrograph of histological sections of the liver stained with Perl's Prussian blue to detect for iron deposits in mice: (top left) untreated NIO, (top center) untreated IO, (top right) IO treated with DFO, (bottom left) IO treated with rPR-OH and (bottom right) IO treated with rPR-DFO (20× original magnification, scale bar=50 μm).

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

The term “alkyl” as used herein is a branched or unbranched hydrocarbon group such as methyl, ethyl, n-propyl, isopropyl, n-butyl, isobutyl, t-butyl, pentyl, hexyl, heptyl, octyl, nonyl, decyl, dodecyl, and the like. The alkyl group can also be substituted or unsubstituted. Unless stated otherwise, the term “alkyl” contemplates both substituted and unsubstituted alkyl groups. The alkyl group can be substituted with one or more groups including, but not limited to, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. An alkyl group which contains no double or triple carbon-carbon bonds is designated a saturated alkyl group, whereas an alkyl group having one or more such bonds is designated an unsaturated alkyl group. Unsaturated alkyl groups having a double bond can be designated alkenyl groups, and unsaturated alkyl groups having a triple bond can be designated alkynyl groups. Unless specified to the contrary, the term alkyl embraces both saturated and unsaturated groups.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is replaced with a heteroatom such as, but not limited to, nitrogen, oxygen, sulfur, selenium or phosphorus. The cycloalkyl group and heterocycloalkyl group can be substituted or unsubstituted. Unless stated otherwise, the terms “cycloalkyl” and “heterocycloalkyl” contemplate both substituted and unsubstituted cyloalkyl and heterocycloalkyl groups. The cycloalkyl group and heterocycloalkyl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. A cycloalkyl group which contains no double or triple carbon-carbon bonds is designated a saturated cycloalkyl group, whereas an cycloalkyl group having one or more such bonds (yet is still not aromatic) is designated an unsaturated cycloalkyl group. Unless specified to the contrary, the term cycloalkyl embraces both saturated and unsaturated, non-aromatic, ring systems.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. A compound depicted with wedges and dashed lines for bonds contemplates both the specifically depicted enantiomer, as well the racemic mixture. The term “enantioenriched” means that the depicted enantiomer is present in a greater amount than the non-depicted enantiomer.

The term “aryl” as used herein is an aromatic ring composed of carbon atoms. Examples of aryl groups include, but are not limited to, phenyl and naphthyl, etc. The term “heteroaryl” is an aryl group as defined above where at least one of the carbon atoms of the ring is replaced with a heteroatom such as, but not limited to, nitrogen, oxygen, sulfur, selenium or phosphorus. The aryl group and heteroaryl group can be substituted or unsubstituted. Unless stated otherwise, the terms “aryl” and “heteroaryl” contemplate both substituted and unsubstituted aryl and heteroaryl groups. The aryl group and heteroaryl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol.

Exemplary heteroaryl and heterocyclyl rings include: benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyL cirrnolinyl, decahydroquinolinyl, 2H,6H˜1,5,2-dithiazinyl, dihydrofuro[2,3b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl.

The terms “alkoxy,” “cycloalkoxy,” “heterocycloalkoxy,” “cycloalkoxy,” “aryloxy,” and “heteroaryloxy” have the aforementioned meanings for alkyl, cycloalkyl, heterocycloalkyl, aryl and heteroaryl, further providing said group is connected via an oxygen atom.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Unless specifically stated, a substituent that is said to be “substituted” is meant that the substituent can be substituted with one or more of the following: alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol.

As used herein, a rotaxane refers to a linear polymer compound encircled by at least one macrocyclic ring, in which each of end of the linear polymer is capped with a sterically large group (a blocking group) to prevent the macrocycle from sliding away from the linear polymer. A macrocycle includes any ring compound in which at least 12 atoms make up the ring circumference. The linear polymer can be designated the skewer. A polyrotaxane refers to a linear polymer compounds encircled by at least two macrocyclic rings, in which each of end of the skewer is capped with a sterically large group to prevent the macrocycles from sliding away from the linear polymer.

Since it is well known that excess iron in cells is extremely dangerous to tissues and organs due to iron-mediated generation of highly toxic Reactive Oxygen Species (ROS) via the Haber-Weiss reaction, one distinctive feature of IO cells is that they are uniquely under more oxidative stress than non-iron overloaded (NIO) cells; this elevated oxidative stress can therefore serve as a selective trigger for degradation of iron-chelating macromolecules to promote their elimination from the body. In contrast to rapidly cleared free DFO, iron-chelating macromolecules are expected to circulate longer and would have the advantage of being able to more efficiently chelate dangerous non-transferrin bound iron (NTBI) present in the circulation, and be able to naturally target the iron storage pool of the liver. Based on an understanding of cellular properties unique to the IO condition, ROS-responsive polyrotaxane-DFO macromolecules were designed to address current challenges associated with free DFO therapy.

Disclosed herein are polyrotaxanes conjugated to metal chelating compounds. The compositions include at least one iron chelating compound conjugated to at least one macrocycle in the polyrotaxane. Because the polyrotaxane is large, it is not readily cleared, and therefore has a prolonged residence time in plasma. At least one of the blocking groups is selectively cleaved in certain chemical environments. Once the blocking group has been cleaved, the individual macrocyclic rings slide off the skewer and can be excreted.

Suitable macrocyclic rings include cyclic polysaccharides, cyclic polypeptides, crown ethers, cucurbiturals, calixarenes, and pillararenes. Cyclic polysaccharides are an especially preferred macrocycle, for instance those having six, seven, or eight sugar units in the ring. In some embodiments, the macrocycle is a cyclodextrin (cyclic 1,4-α-D-glucopyranosides) such as α-cyclodextrin (6 carbohydrate units), β-cyclodextrin (7 carbohydrate units), or γ-cyclodextrin (8 carbohydrate units).

The metal chelator can be a compound that strongly binds one or more metals. In a preferred embodiment, the metal chelator strongly binds to iron (II) or iron (III), or both iron (II) and iron (III). Suitable chelators include deferoxamine, desferasirox, deferiprone, which can be covalently attached to at least one macrocyclic ring. Such macrocycles are designated chelator-functionalized macrocycles. In certain embodiments, each chelator-functionalized macrocycle will bear an average of one, two, or three metal chelator groups. For embodiments in which the macrocycles are polysaccharides, for instance, cyclodextrin, the metal chelator can be bound through the primary hydroxyl group, i.e., at the ‘6’ carbon. The metal chelator can be functionalized with an isocyanate, and allowed to react with the primary alcohol in the cyclodextrin. Alternatively, metal chelators bearing nucleophilic groups, e.g., amine groups, can be conjugated by first activating the cyclodextrin primary alcohol with an electrophilic carbonyl (e.g., with carbonyl-diimidazole) and then reacting the activated cyclodextrin with the metal chelating compound.

The polyrotaxanes can also include one or more macrocycles that are conjugated to a solubilizing enhancer. Such macrocycles are designated solubilizer-functionalized macrocycles. Exemplary solubilizing group include primary alcohols, amines, carboxylates, sulfonates, phosphonates, and pharmaceutically acceptable salts thereof. In certain embodiments, each solubilizer-functionalized macrocycle will bear an average of one, two, or three solubilizer groups.

For embodiments in which the macrocycles are polysaccharides, for instance, cyclodextrin, the solubilizing group can be bound through a short carbon chain via the primary hydroxyl group at the ‘6’ carbon.

The polyrotaxanes can also include one or more macrocycles that are conjugated to a radionuclide, e,g., a radioactive atom such as used in positron emission tomography and/or radiation therapy. Exemplary radionuclides include ¹⁸F, ¹¹C, ¹²³I, ¹²⁴I, ¹²⁷I, ¹³¹I, ⁷⁶Br, ⁶⁴Cu, ⁹⁹Tc, ⁹⁹Y, ⁶⁷Ga, ⁵¹Cr, ¹⁹²Ir, ⁹⁹Mo, ¹⁵³Sm, ²⁰¹Tl, ⁷²As, ⁷⁴As, ⁷⁵Br, ⁵⁵Co, ⁶¹Cu, ⁶⁷Cu, ⁶⁸Ga, ⁶⁸Ge, ¹²⁵I, ¹³²I, ¹¹¹In, ⁵²Mn, ²⁰³Pb, and ⁹⁷Ru. The radionuclide may be conjugated to the macrocycle using a compound having the formula Z—(CH₂)ₙ—Y, wherein Y is the radionuclide, n is from 1-10, and Z is an isocyanate or primary amine. In some embodiments, the polyrotaxanes can include one or more macrocycles that are conjugated to a fluorescent tracer. Suitable tracers and techniques for conjugating them to primary alcohols such as found in a cyclodextrin are well-understood by the skilled person. Exemplary fluorescent tracers include carbocyanines, aminostyryl, rhodamine, 5-chloromethylfluoresceines, 4-halo-methylcoumarins, Lucifer yellows, stillbamidines, 8-halomethylboa-diazas-indacene, quantum dots, and fluorescent microspheres.

In certain embodiments, the polyrotaxane will also include macrocycles that are not conjugated to either a metal chelating agent or solubilizing enhancer. Such macrocycles are designated unfunctionalized macrocycles.

Suitable skewers include polymers of ethylene glycol, glycolic acid, lactic acid, vinyl alcohol, and copolymers thereof. The polymer can be a poly(alkylene glycol), polyester, polycarbonate, polyvinyl alcohol, polyanhydride, polyacetal, or a combination or copolymer thereof. Preferred polymers include polyethylene glycol (PEG) and polyvinyl alcohol. For instance, the skewer can be a PEG having a M_{w} from 500-50,000 Da, from 1,000-50,000 Da, from 1,000-30,000 Da, from 2,000-30,000 Da, from 2,000-25,000 Da, from 2,000-15,000 Da, from 2,000-10,000 Da, from 2,000-5,000 Da, from 5,000-20,000 Da, from 5,000-10,000 Da, from 10,000-50,000 Da, or from 25,000-50,000 Da.

The blocking group at each of the skewer can be a biocompatible, sterically large group that prevents the macrocycle from dissociating from the skewer. Suitable blocking groups include adamantyl groups, triphenylmethyl groups, cyclodextrins, amino acids and oligopeptides, and pyrenes.

The blocking group can be associated with the skewer via selectively cleavable linkers. Selectively cleavable linkers are those that undergo bond dissociation in specific chemical environments. For instance, linkers that are unstable to free radicals will collapse in the presence of reactive oxygen species. Such linkers are designated ROS sensitive linkers. Exemplary ROS sensitive functional groups include thioacetals, oxalate esters, peptides, and diselenide (—Se—Se—) groups. Linkers that collapse in acidic or basic medium include Schiff base/imine groups, boronic esters and acetals. Some linkers may feature both ROS and pH sensitive functionalities.

In some embodiments, the polyrotaxane may be have the formula:

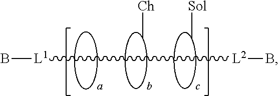

wherein [symbol] represents a linear polymer,

[symbol] represents a macrocycle, and a is selected from 0-250; 0-100; 0-50; 1-40; 2-40; 5-40; 10-40; 20-40; 1-5; 1-10; 1-20; or 5-20;

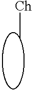

represents a macrocycle conjugated with at least one metal chelator, and b is selected from 1-250; 1-100; 1-50; 1-40; 2-40; 5-40; 10-40; 20-40; 1-5; 1-10; 1-20; or 5-20;

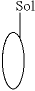

represents a macrocycle conjugated with at least one solubilizing group, and c is selected from 0-250; 0-100; 0-50; 1-40; 2-40; 5-40; 10-40; 20-40; 1-5; 1-10; 1-20; or 5-20;

L¹ and L² are each linkers, provided at least one of L¹ or L² is a cleavable linker, and

B is a blocking group,

wherein the macrocycle, metal chelator, solubilizing group, linker and blocking group are as defined above.

It is to be understood that the occurrence of the chelator-functionalized macrocycles, solubilizer-functionalized macrocycles, and unfunctionalized macrocycles can be random along the skewer, and any particular depiction of a polyrotaxane is not intended to convey any particular sequence of macrocycles along the skewer. Such polyrotaxanes may further include radionuclide or tracer functionalized macrocycles.

In preferred embodiments, the polyrotaxane have the formula:

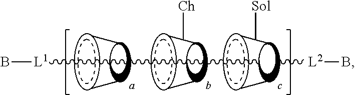

wherein [symbol] represents a linear polymer,

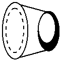

represents an unfunctionalized cyclodextrin, and a is selected from 0-250; 0-100; 0-50; 1-40; 2-40; 5-40; 10-40; 20-40; 1-5; 1-10; 1-20; or 5-20;

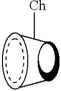

represents a cyclodextrin conjugated with at least one metal chelator, and b is selected from 1-250; 1-100; 1-50; 1-40; 2-40; 5-40; 10-40; 20-40; 1-5; 1-10; 1-20; or 5-20;

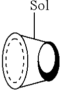

represents a cyclodextrin conjugated with at least one solubilizing group, and c is selected from 0-250; 0-100; 0-50; 1-40; 2-40; 5-40; 10-40; 20-40; 1-5; 1-10; 1-20; or 5-20;

L¹ and L² are each linkers, provided at least one of L¹ or L² is a cleavable linker, and

B is a blocking group wherein the cyclodextrin, metal chelator, solubilizing group, linker and blocking group are as defined above. Such polyrotaxanes may further include radionuclide or tracer functionalized cyclodextrins.

In some embodiments,

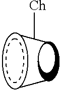

represents a macrocycle having the formula:

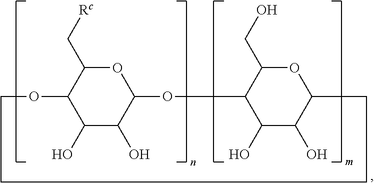

wherein n is selected from 1-8, m is selected from 0-7, wherein the sum of n+m is 6, 7 or 8; and

R^{c} is—is selected from —O—X¹—Z, —NHC(═O)—X¹—Z, OC(═O)NH—X¹—Z, wherein X¹ is absent, aryl, alkaryl, or (CH₂)ₓ, wherein x is selected from 1-10 and Z is a metal chelator. Preferred n values are 1 and 2.

In some cases,

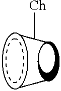

represents a cyclodextrin having the formula:

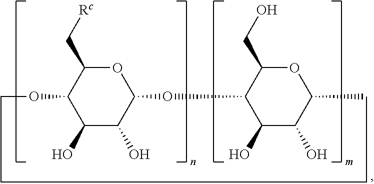

wherein n, m and R^{c} have the meanings above.

Preferred R^{c} moieties include:

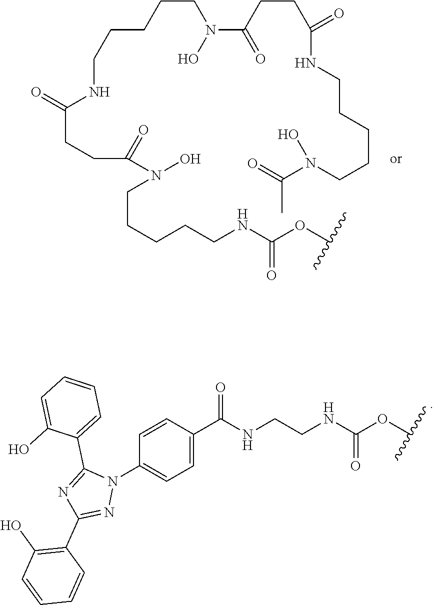 [SMILES: *OC(=O)NCCCCCN(O)C(=O)CCC(=O)NCCCCCN(O)C(=O)CCC(=O)NCCCCCN(O)C(C)=O ; *OC(=O)NCCNC(=O)c1ccc(-n2nc(-c3ccccc3O)nc2-c2ccccc2O)cc1]

In some embodiments,

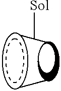

represents a cyclodextrin having the formula:

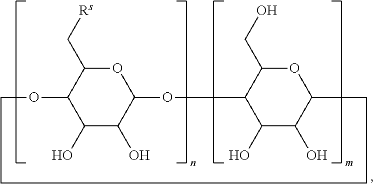

wherein n is selected from 1-8, m is selected from 0-7, wherein the sum of n+m is 6, 7 or 8; and

and R^{s} is—is selected from —O—(CH₂)_{y}—Y, —NH—(CH₂)_{y}—Y, —NHC(═O)—(CH₂)_{y}—Y, OC(═O)NH—(CH₂)_{y}—Y, preferably OC(═O)NH—(CH₂)_{y}—Y, wherein y is selected from 1-4, preferably 2, and Y is selected from OH, NH₂, COOH, SO₃H, or PO₃H₂, preferably OH. Preferred n values are 1 and 2.

In some cases

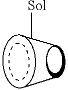

represents a cyclodextrin having the formula:

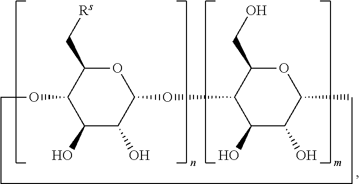

and n, m, and R^{s} have the meanings given above.

B can have the formula:

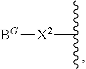

wherein X² is absent, —NH—, —O—, —C(═O)NH—, —NHC(═O)NH—, —NHC(═O)O—, —OC(═O)NH—, —NHC(═O)—; and B^{G} is adamantyl, triphenylmethyl, a cyclodextrin.

It is preferred that the L¹ and L² groups are the same. Suitable L¹ and L² groups include:

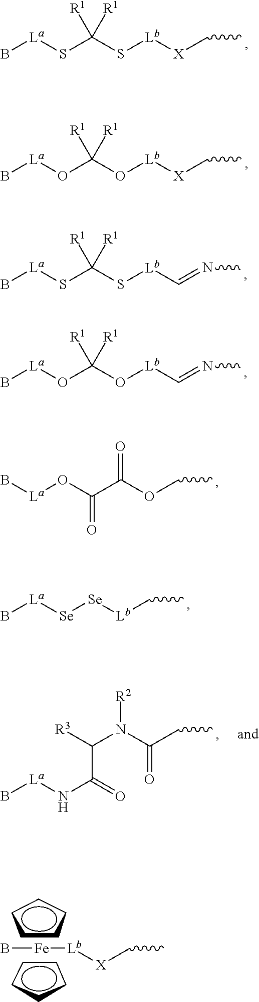

wherein X is O, NH, S, or absent;

R¹, R² and R³ are in each case independently selected from H, C₁₋₈alkyl, aryl, and any two of R¹, R², and R³ may together form a ring;

L^{a} is —(CH₂)_{z}, wherein z is 0-10, e.g., 0, 1, 2, 3, 4, 5, 6, 7, 8, 9, or 10; and

L^{b} is —(CH₂)_{y}, wherein y is 1-10, e.g., 0, 1, 2, 3, 4, 5, 6, 7, 8, 9, or 10.

The polyrotaxanes disclosed herein may be prepared by combining a skewer compound with a macrocyclic compound in a solvent. The ends of the skewer compound will typically be a reactive functional group like a carboxylic acid, a primary amine, or a thiol. After a suitable amount of time, a blocking group reagent can be added. In some embodiments, the macrocycle is functionalized with the chelating agent and solubilizing enhancer prior to threading with the skewer, while in other cases the chelating agent and solubilizing are conjugated to the macrocycles after the polyrotaxane has been prepared. Cyclodextrin compounds may be selectively activated at the primary alcohol using a reagent like carbonyldiimidazole, and then reacted with an amine-bearing chelating compound, followed by an amine-bearing solubilizing enhancer, for instance, 2-aminoethanol.

The polyrotaxanes disclosed herein may be administered to a patient in need thereof by IV injection or infusion, in a suitable physiological solution. A liquid pharmaceutical composition may include one or more of the following carriers or excipients: sterile diluents such as water for injection, saline solution, preferably physiological saline, Ringer's solution, isotonic sodium chloride, fixed oils such as squalene, squalene, mineral oil, a mannide monooleate, cholesterol, and/or synthetic mono or digylcerides, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The composition can be enclosed in ampoules, disposable syringes or multiple dose vials made of glass or plastic. An injectable pharmaceutical composition is preferably sterile

Pharmaceutical compositions (may also contain diluents such as buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents. Preferably, product may be formulated as a lyophilizate using appropriate excipient solutions (e.g., sucrose) as diluents.

### EXAMPLES

The following examples are for the purpose of illustration of the invention only and are not intended to limit the scope of the present invention in any manner whatsoever.

### Example 1: Synthesis of Synthesis of ROS-cleavable Thioketal End-Caps (TEC)

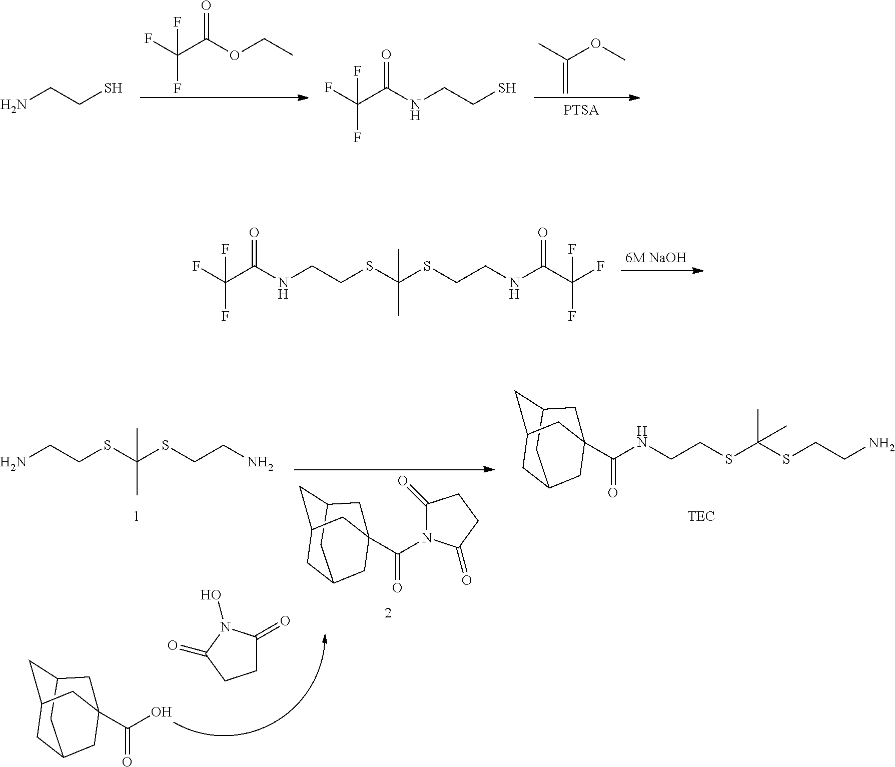

ROS-cleavable thioketal linker (Compound 1) and 1-(N-succinimidyloxycarbonyl) adamantine (Compound 2) was synthesized following a reported method. To a suspension of Compound 1 (388 mg, 2 mmol) in CH₂Cl₂ (5 mL) was dropwise added Compound 2 (277 mg, 1 mmol) in CH₂Cl₂ (10 mL) at RT. The resulting solution was stirred at room temperature for 24 h. The solvent was removed under reduced pressure. Flash chromatography over silica gel column (1/40-1/10 MeOH/CH₂Cl₂) provided TEC as a slight yellow solid.

### Example 2: Synthesis of rPR-DFO

PEG 4000 (2.52 g, 0.63 mmol) were oxidized in 100 mL of water with TEMPO (210 mg, 1.34 mmol), NaBr (220 mg, 2.14 mmol), and 10 mL aqueous NaClO at pH 10-11 at room temperature for 15 min. The oxidation was quenched by the addition of 10 mL of ethanol, acidified with HCl to pH<2 and then extracted three times with 100 mL of CH₂Cl₂. CH₂Cl₂ layers were dried under reduced pressure and PEG-COOH was obtained by recrystallization with ethanol at −20° C. overnight. PEG-COOH (1 g, 0.25 mmol) was added dropwise to 100 mL of saturated aqueous solution of α-CD. The mixture was ultrasonicated for 15 min and stirred at room temperature overnight, giving white paste-like pseudorotaxanes (pPR). The obtained pseudorotaxanes were freeze-dried for 24 h and dissolved in 10 mL of anhydrous DMF and mixed with TEC (926 mg, 2.6 mmol), BOP reagent (960 mg, 2.2 mmol), and EDIPA (380 mg, 2.4 mmol). The mixture was allowed to react at 4° C. overnight, followed by washing two times with DMF/methanol (1:1), two times with methanol, and two times with water by centrifugation. rPR was obtained as a white solid after freeze-drying for 24 h.

rPR (500 mg) was dissolved in 50 mL of anhydrous DMSO under nitrogen and then added dropwise to CDI (carbonyl di-imidazole) (10.0 g) in 50 mL of anhydrous DMSO under nitrogen. The mixture was stirred at room temperature for 24 h and then was poured into 900 mL of tetrahydrofuran/diethyl ether (1:2) mixed solvent to precipitate the product (CDI-modified rPR), followed by washing two times with THF/diethyl ether (1:1) mixed solvent. The resulting solid were mixed in 50 mL of anhydrous DMSO with DFO (328 mg, 0.5 mmol) and EDIPA (130 mg, 1 mmol) at room temperature for 24 h and then added dropwise to ethanolamine (20 mL) in 20 mL of DMSO under nitrogen. After stirring at room temperature for another 24 h, the reaction mixture was poured into 500 mL of THF/diethyl ether (2:1) mixed solvent to precipitate rPR-DFO. The aqueous solution of rPR-DFO was dialyzed with dialysis tubing (MWCO 10 KDa) for 3 d and then freeze-dried to obtain the final product.

rPR-OH was synthesized similarly without the addition of DFO.

SulfoCy-5.5 (5.4 mg, 0.005 mmol) was added to a 5 mL anhydrous solution of DMSO containing CDI-modified rPR (50 mg), DFO (32.8 mg, 0.05 mmol) and DIPEA (13 mg, 0.1 mmol). The resulting mixture was allowed to stir at room temperature in the dark for 24 h and subsequently added dropwise to a solution of 2 mL ethanolamine in 2 mL of DMSO under nitrogen for another 24 h at room temperature. The reaction mixture was dialyzed against water with dialysis tubing (MWCO 10 kDa) for 3 days and then freeze-dried to obtain the final SCy5.5-rPR-DFO.

¹H NMR, 2D DOSY and 2D ROESY spectra were acquired with a 400 MHz Bruker NMR spectrometer. Iron chelation property and amount of DFO conjugated to rPR-DFO were quantified by monitoring the UV/Vis absorption spectra in the presence of iron(III) by scanning between 400-700 nm with a SpectraMax Plus spectrophotometer (Molecular Devices). The magnitude of the absorbance peak at 430 nm, which is characteristic of the complex concentration in solution when DFO chelates to iron(III) at a 1:1 ratio, was used to characterize the degree of complexes formed. The standard curve and equation to calculate DFO concentration based on absorption was conducted as previously reported.⁴ To investigate the morphology of rPR-DFO, transmission electron microscopy (TEM) images were taken on a JEM1011 instrument at an acceleration voltage of 100 kV. Hydrodynamic size and polydispersity (PDI) of rPR-DFO were measured by dynamic light scattering (DLS) with a Zetasizer Nano ZS (Malvern Instruments, UK) and analyzed with Zetasizer software v7.10. Measurements were conducted on three batches of samples and results are reported as mean±standard deviation (SD).

2D rotating-frame Overhauser effect spectroscopy (2D ROESY) of rPR shows the clear crosspeaks belonging to the nonbonding interaction between H-3 (δ=3.72 ppm) and H-5 (δ=3.64 ppm) protons of α-CD and the CH₂ (δ=3.51 ppm) of PEG, indicating that PEG axle threaded into the cavity of CD rings. Further, to demonstrate the structural stability of the rPR, two-dimensional diffusion-ordered NMR spectroscopy (2D DOSY) spectrum of rPR was compared with that of PPR. Each molecule only has one diffusion coefficient (D) value which varies with different molecular weights. Two separate peaks corresponding to α-CD and PEG respectively were observed along the diffusion axis in the 2D DOSY spectrum for PPR.

The proton signals of α-CD and PEG are associated with different D value of about 8.13×10-11 m²/s and 3.98×10-10 m²/s respectively, suggesting that the CDs dethread from the axis of PEG in DMSO and PPR separated into two components with different molecular weight. In contrast, The 2D DOSY spectrum for rPR shows that all proton peaks from α-CD and PEG exhibits the same D of approximately 1.99×10-11 m²/s and only a single peak corresponding to the D value can be found along the diffusion axis. These results reveal that the supramolecular structure of rPR is stable as a whole unit.

The degradation of rPR-DFO under simulated ROS conditions was characterized by ¹H NMR spectroscopy. The proton signals from rPR-DFO are broad and unresolved, indicating that the supramolecular architecture of rPR-DFO is stable in H₂O and the rotaxanation decreases remarkably the conformational flexibility of CD and PEG moieties, causing the broadening of the signals. In contrast, because of the efficient cleavage of TEC by ROS, rPR-DFO is not stable in H₂O and CD moieties dethread from the polymer axle to yield a mixture of separated CDs and PEG, leaving their proton signals narrow. ¹H NMR spectrum also clearly confirms the generation of acetone (a relevant degradation product of thioketal) at δ=2.10 ppm, which is in agreement with a previous study regarding the dethioacetalization using H₂O₂.

### Example 3: Evaluation of a rPR-DFO Conjugate

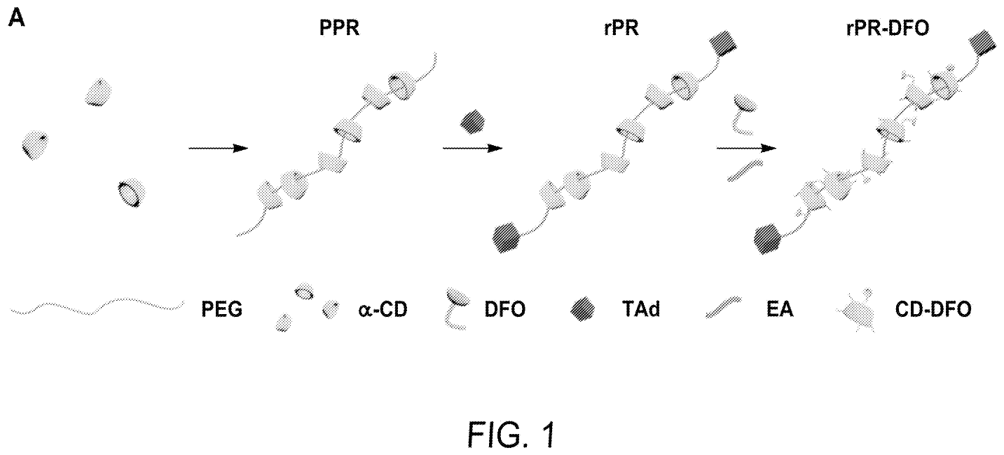

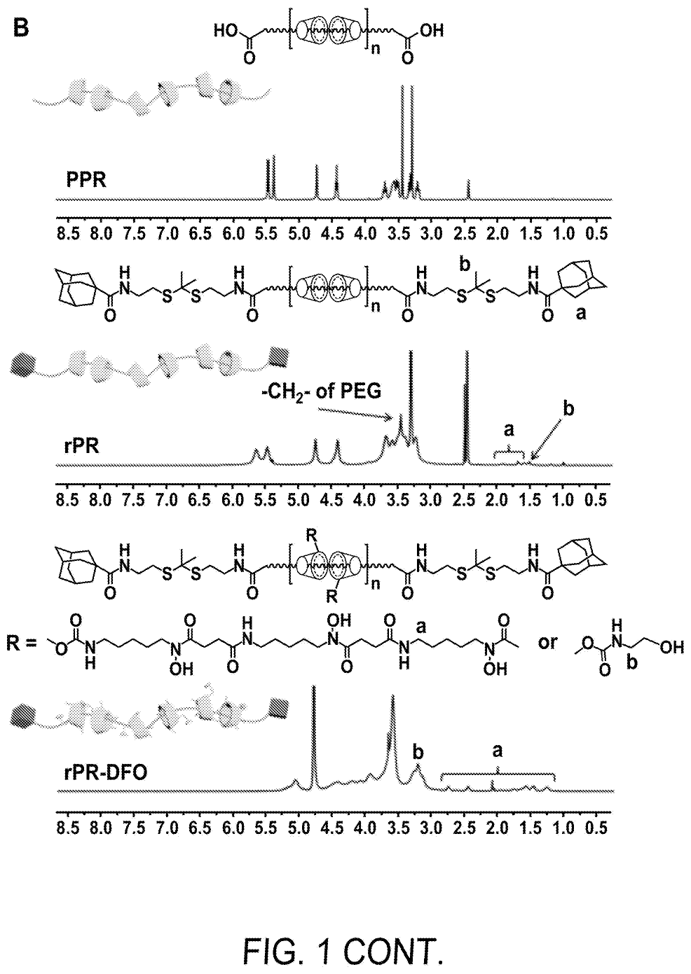

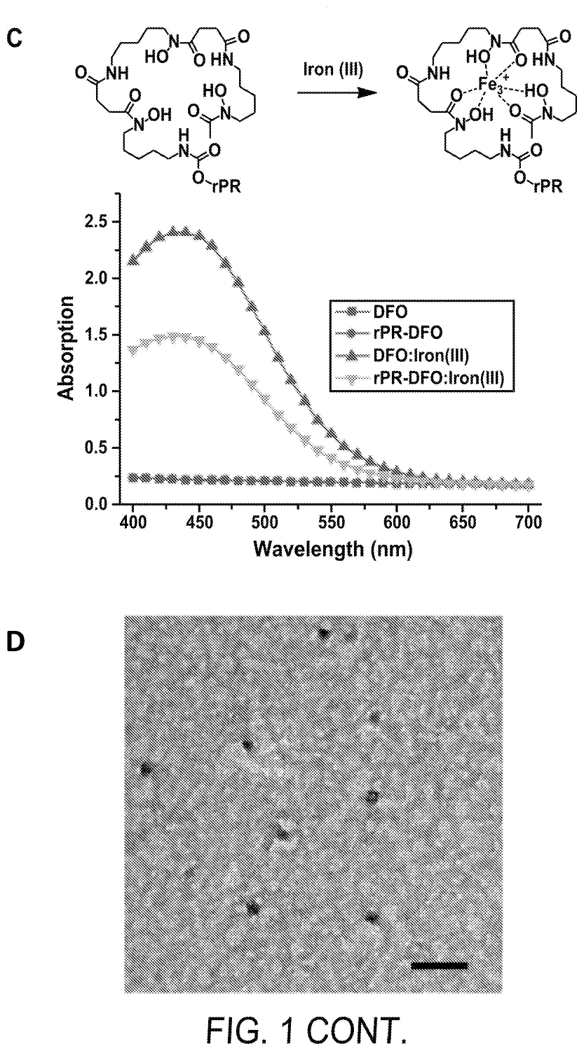

The iron chelating capability and DFO incorporation into the polyrotaxane were determined by monitoring the UV/Vis absorption spectra in the presence of iron(III) by scanning between 400-700 nm with a SpectraMax Plus spectrophotometer (Molecular Devices). The magnitude of the absorbance peak at 430 nm, which is characteristic of the complex concentration in solution when DFO chelates to iron(III) at a 1:1 ratio, was used to characterize the degree of complexes formed (FIG. 1).

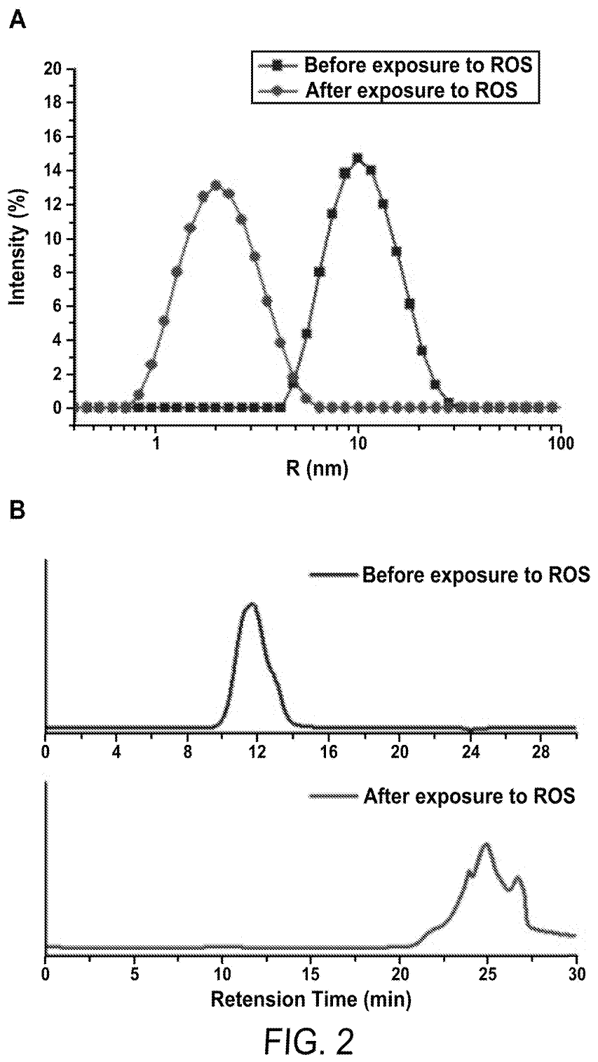

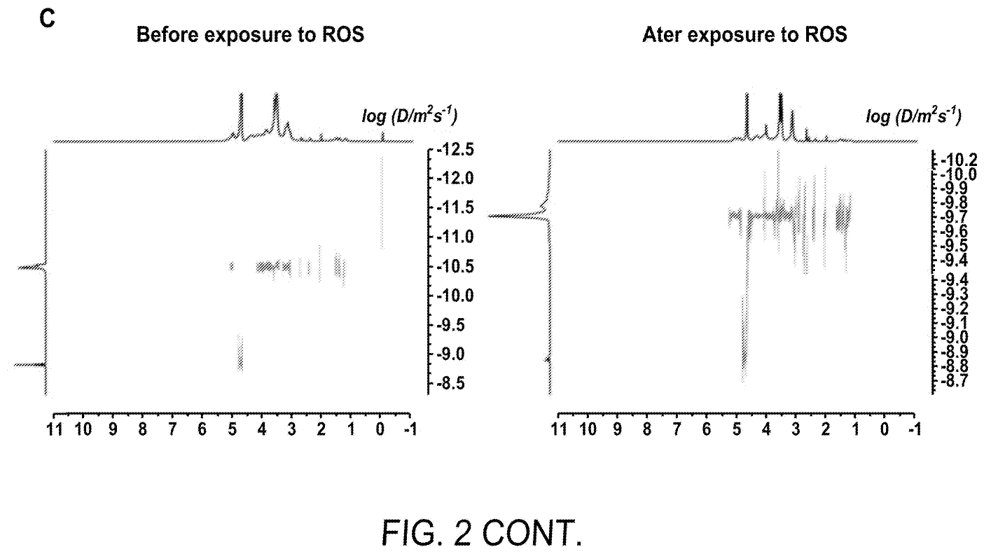

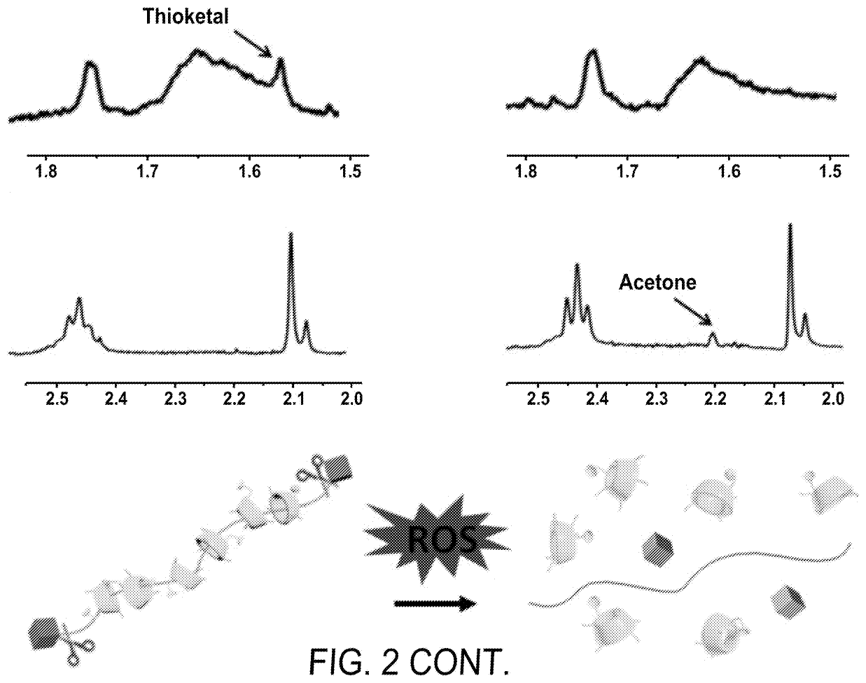

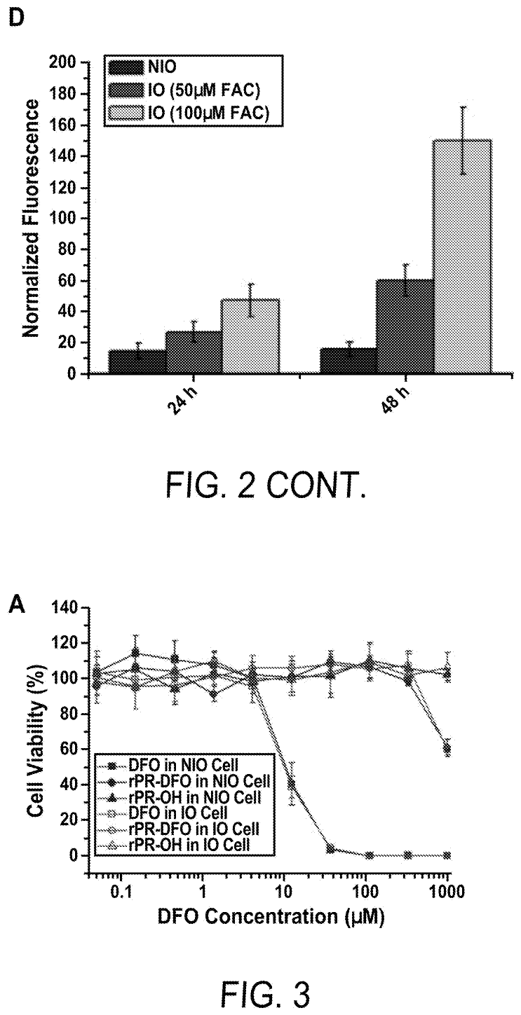

The morphological structure of rPR-DFO was examined by transmission electron microscopy (TEM). Representative TEM micrographs revealed near-spherical shapes of rPR-DFO with diameters of ca. 10.0 nm for their random coil behaviors in good solvents which is consistent with previous works (FIG. 2). Dynamic light scattering (DLS) displayed a single peak that was characterized by a z-average diameter of 9.957±0.0395 nm with a PDI of 0.13±0.002, indicating that rPR-DFO are reasonably monodisperse.

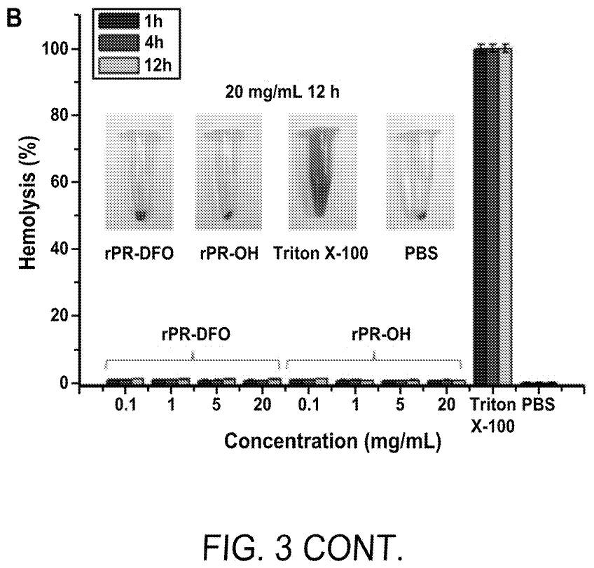

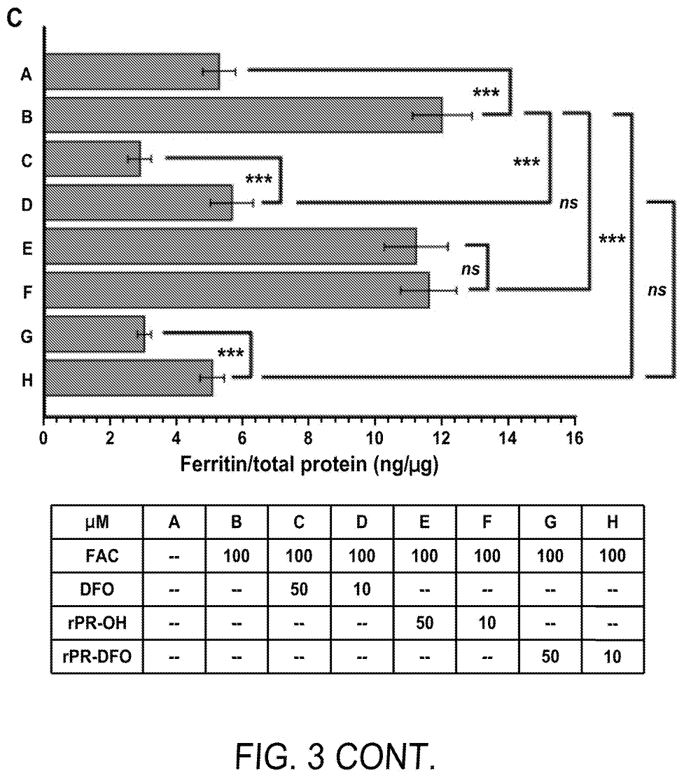

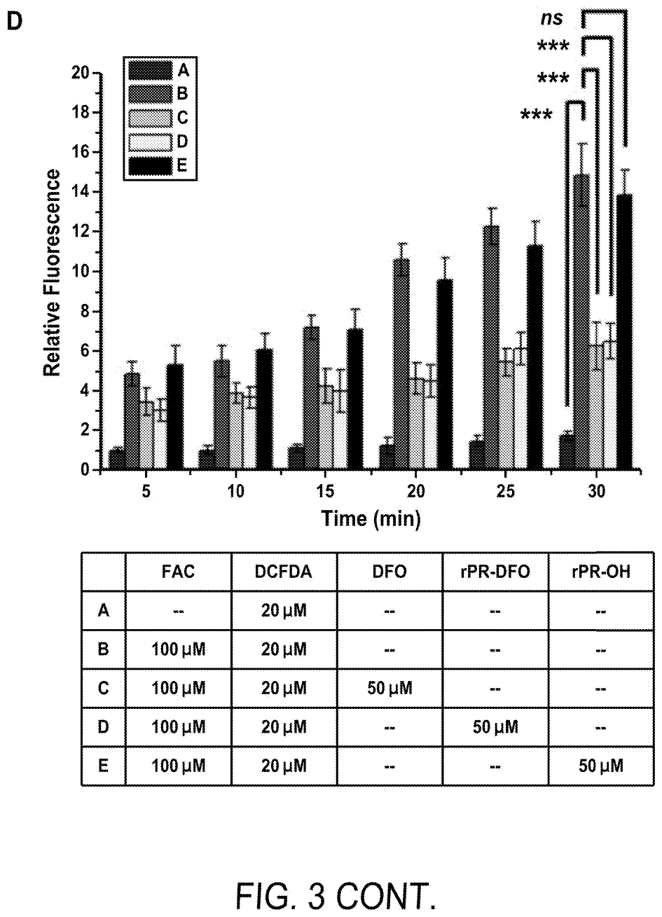

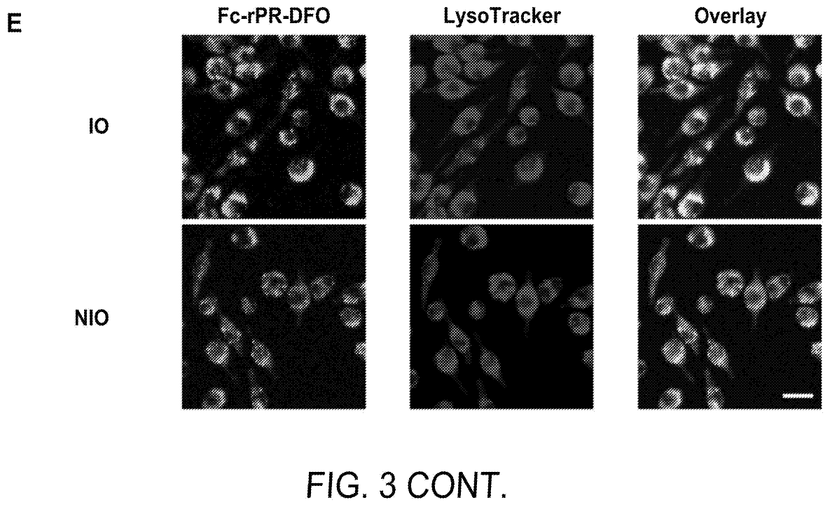

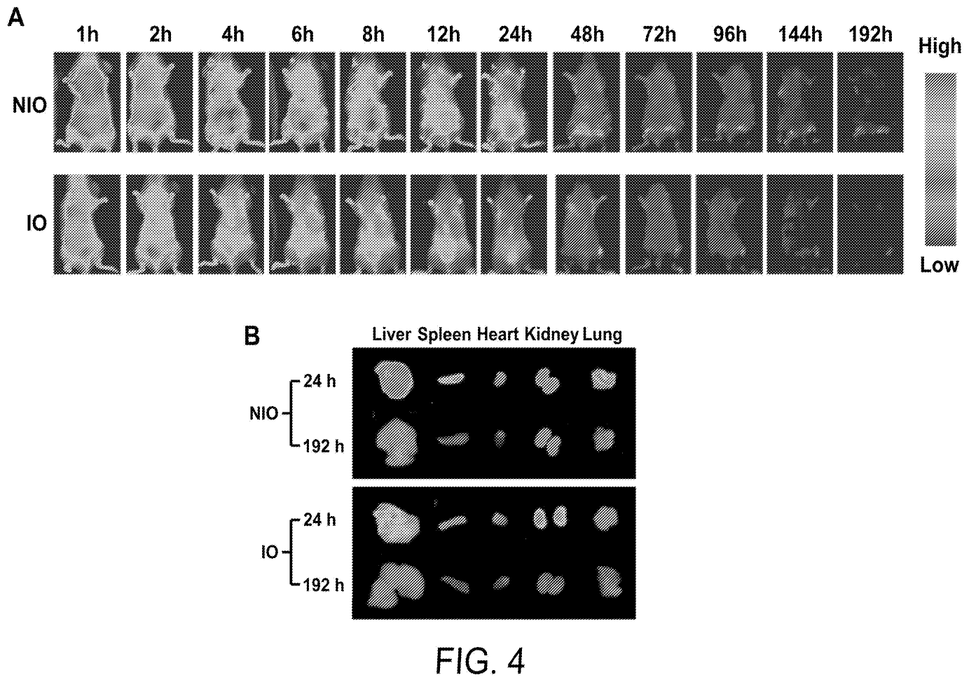

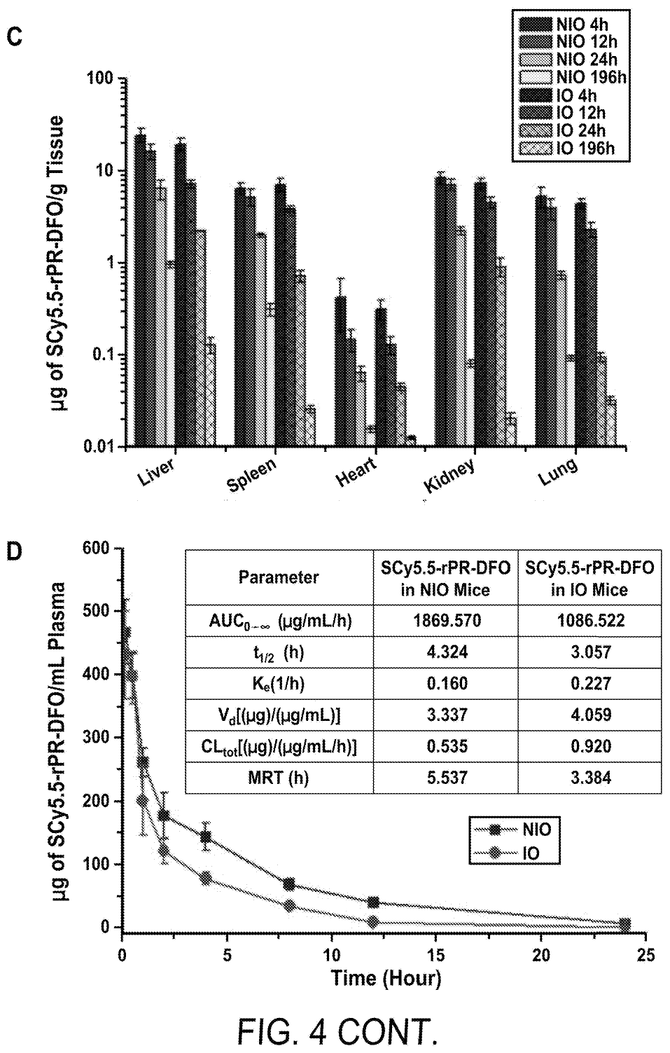

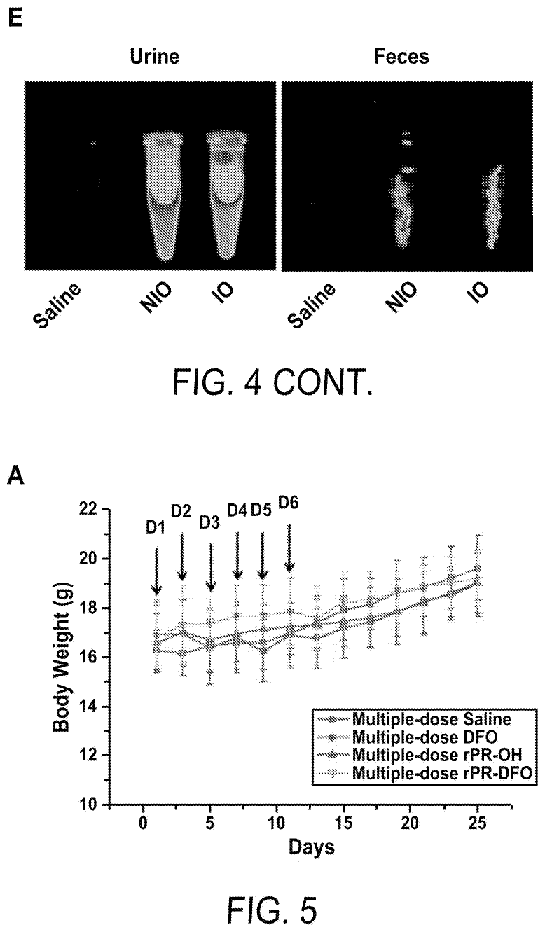

The in vitro degradation size change of rPR-DFO in the presence of 100 μM H₂O₂ was monitored with DLS. rPR-DFO dissociated into smaller degradation fragments after 24 h of incubation in the oxidative environment and the z-average diameter decreased from ca. 9.957±0.0395 nm to ca. 2.615±1.065, a size below the reported renal threshold of 5-10 nm for macromolecule elimination from the body (FIG. 3). The decrease in molecular weight of rPR-DFO after incubation with 100 μM H₂O₂ was also confirmed by GPC, indicative of CDs threading off the PEG chains in response to ROS cleavage of the end-caps (FIG. 4).

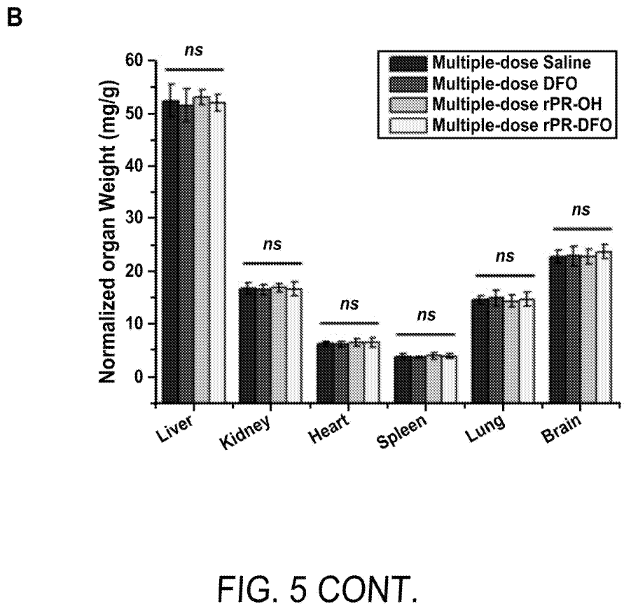

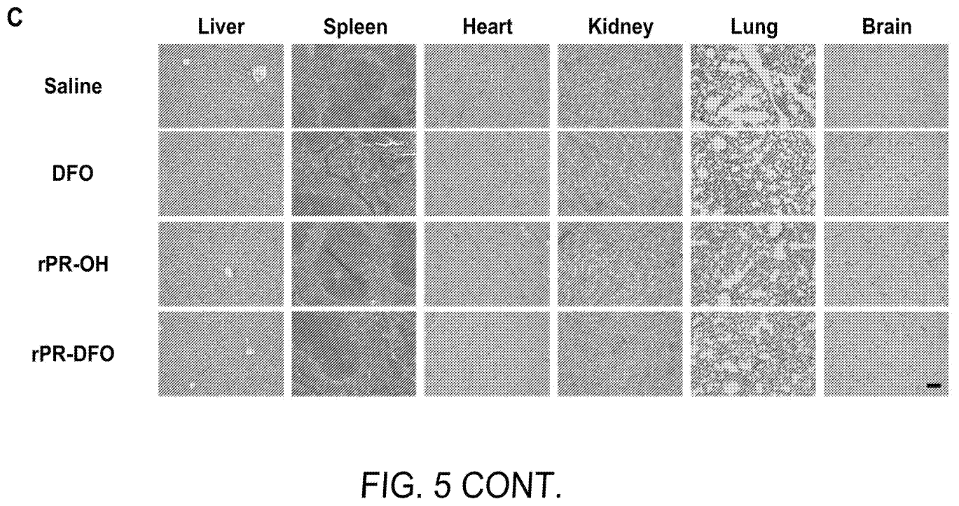

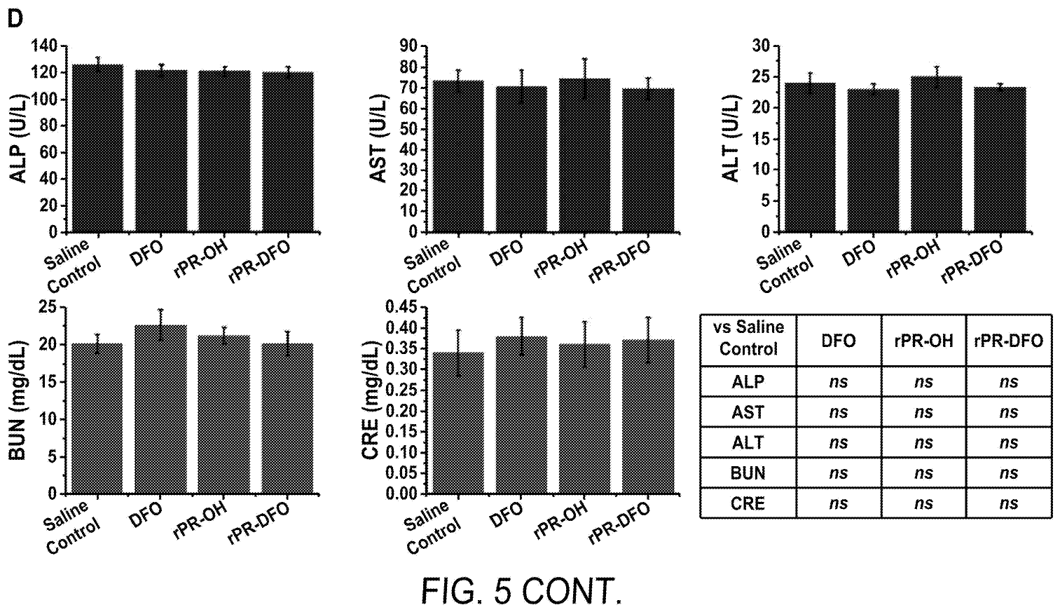

To study the degradation behavior in cells, non-iron overloaded (NIO) and iron overloaded (IO) cells were treated with fluorescein cadaverine (Fc) labeled rPR-DFO in DMEM complete medium solutions. After 24 or 48 h time points, medium was collected from wells and washed with a centrifugal filtration unit (MWCO 10,000). The fluorescence change in the flowthrough (filtrate) was measured at the indicated times by exciting at 493 nm and measuring the emission at 517 nm at 37° C. As shown in FIG. 5, the fluorescence intensity from the filtrate of IO cells medium was significantly higher compared to that of NIO cells after 24 h and 48 h. This indicates that degradation of rPR-DFO by IO macrophage cells is time-dependent, as demonstrated by increased excretion of Fc-labeled degradation products from cells into the medium at 48 h compared to 24 h.

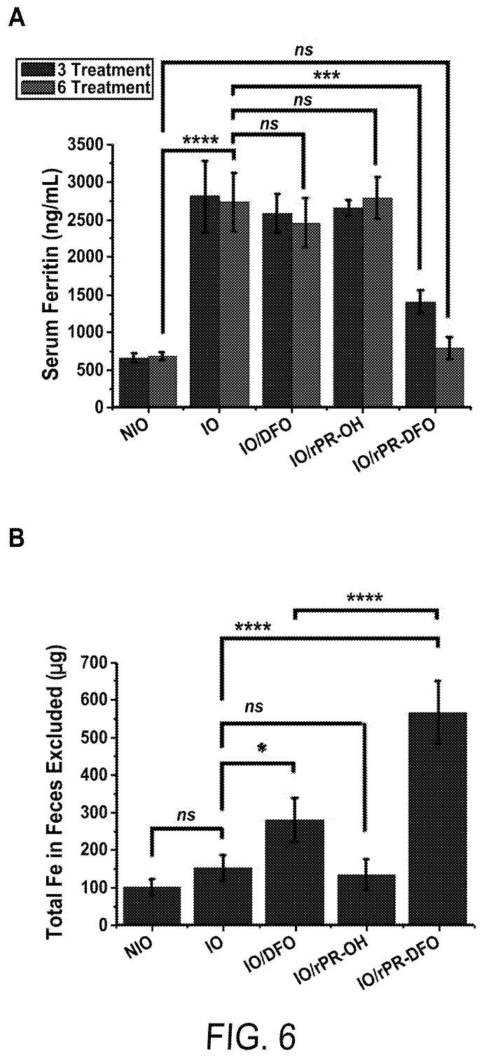

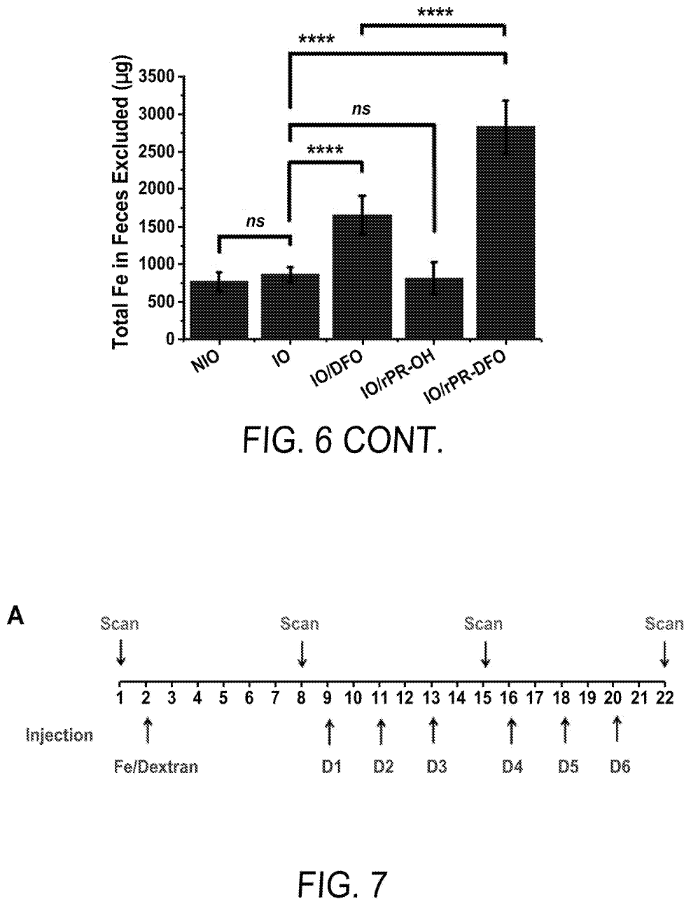

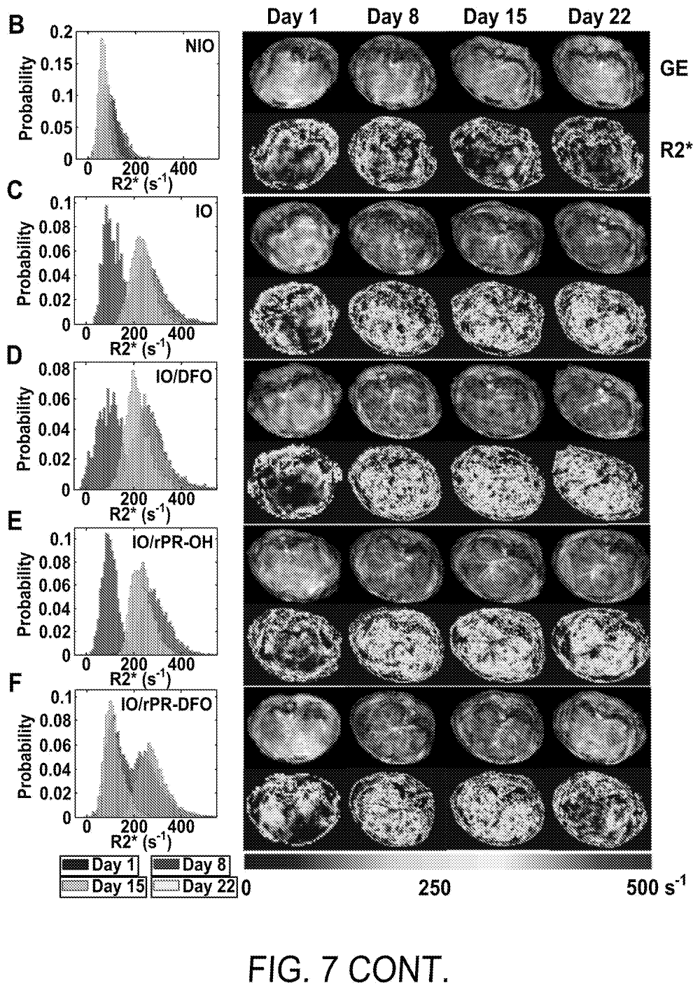

The degradation of rPR-DFO chelated to iron(III) was monitored by incubating the constructs with 100 uM H₂O₂ as the oxidizing agent and monitoring the degradation products by UV-vis. After 24 h incubation, the rPR-DFO:iron(III) sample was washed with a centrifugal filtration unit (MWCO 10,000) and both the recovered concentrate and the filtrate were monitored with UV/Vis scanning at 400-750 nm. Small degradation products consisting of CD-DFO:iron(III) complexes easily passed through the membrane filter and a signal was detected in both the top concentrate and bottom filtrate. However, in the absence of the oxidizing agent, rPR-DFO:iron(III) could not pass through the filter and a signal was only observed in the concentrate and not the filtrate (FIG. 6). In non-oxidative environments, DLS data revealed that rPR-DFO is stable and no degradation products were observed when incubated up to 240 hours at pH 5, 7.4, and 10 (FIG. 7).

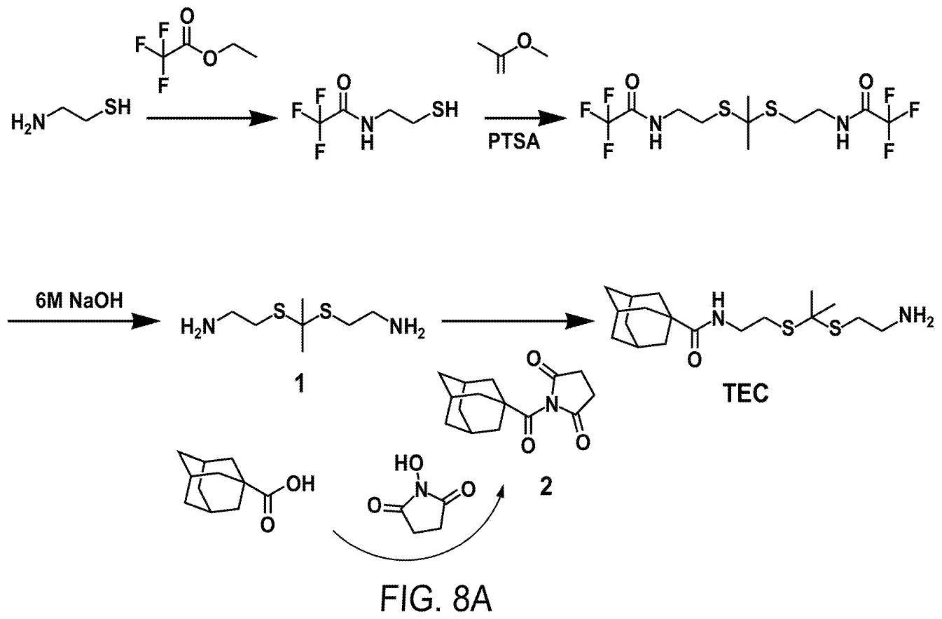

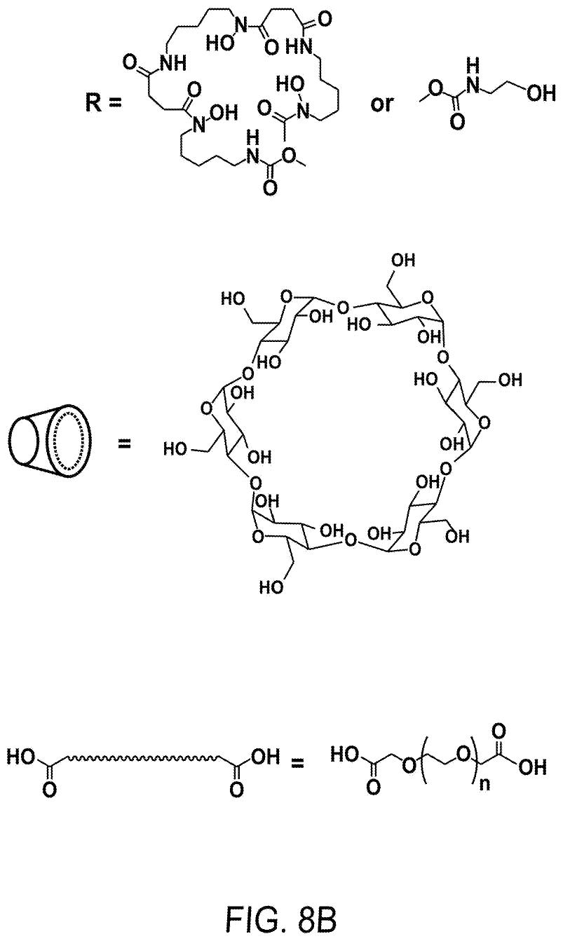

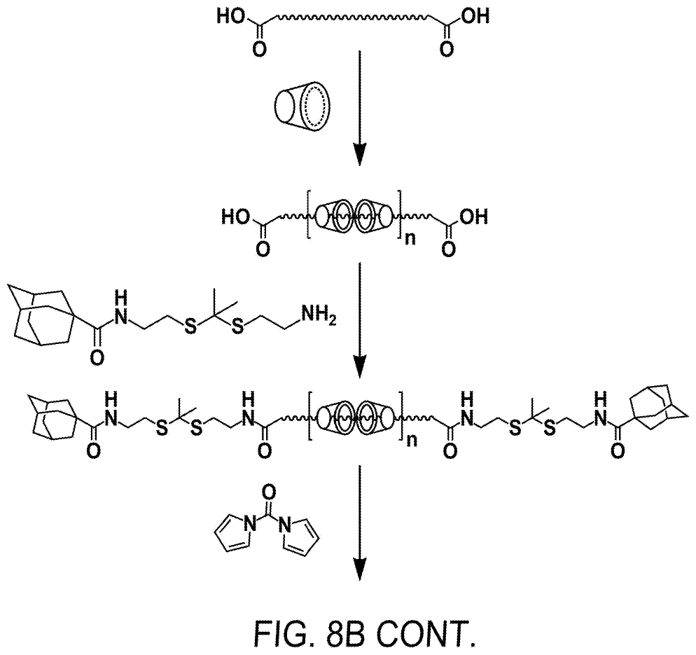

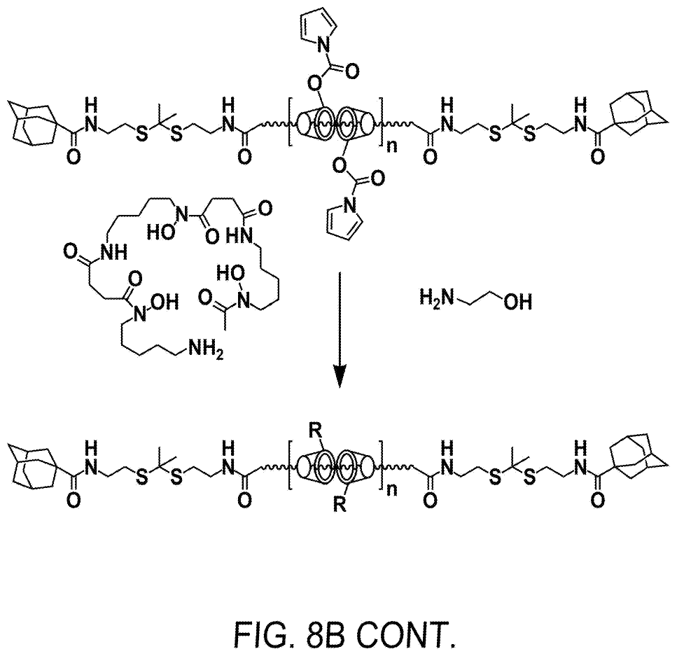

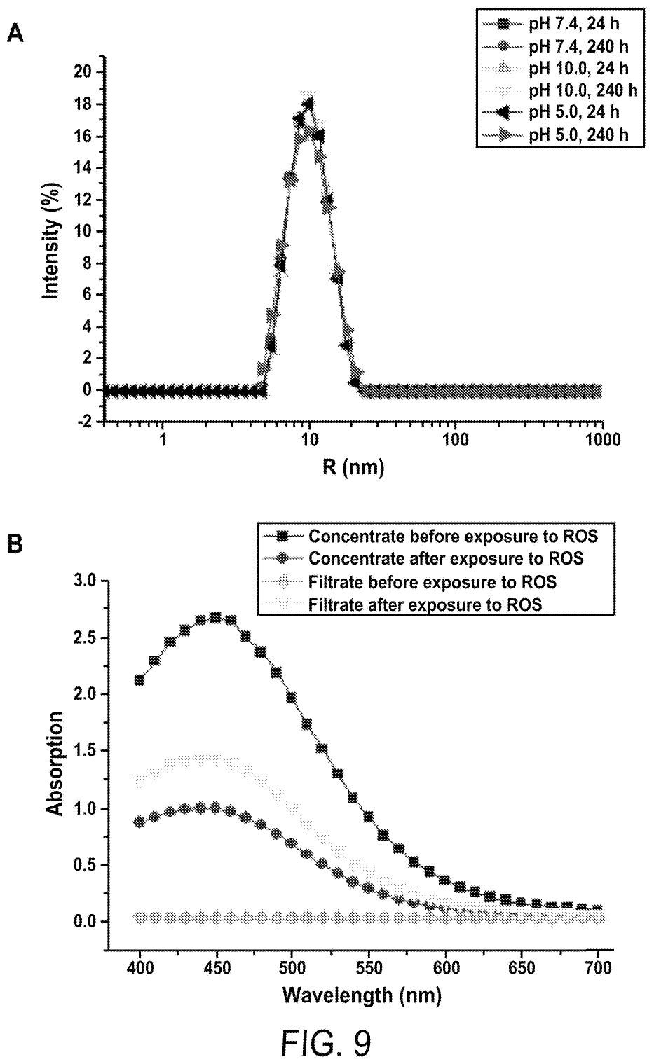

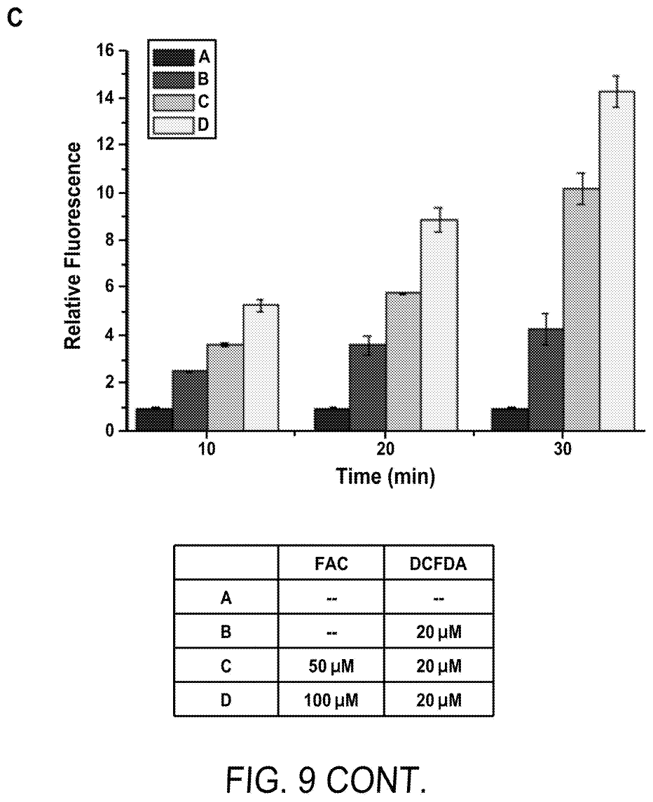

The in vitro cytotoxicity of rPR-OH, rPR-DFO and DFO was evaluated with the metabolism-based resazurin assay on both NIO (FIG. 8) and IO macrophage cells (FIG. 9). IO macrophage cells were iron overloaded for 24 h by incubating with culture medium containing 100 μM ferric ammonium citrate (FAC) prior to addition of formulations and evaluation of cytotoxicity as described below. Briefly, both NIO and IO J774A.1 macrophage cells were seeded in 96-well plates at a density of 3,000 cells/well, cultured at 37° C., 5% CO₂ and 100% humidity with DMEM complete medium for 24 h. Cells were then treated with 1 mM free DFO or rPR-DFO at equivalent DFO concentrations (prepared by 1:3 serial dilutions) for 48 h; equivalent rPR-OH based on w/v to rPR-DFO concentrations was also investigated. Next, the substrate resazurin was dissolved in cell culture medium at a concentration of 44 μM, added to each well (100 μl), and incubated at 37° C. for 4 h. The fluorescence change was monitored by exciting at 560 nm and measuring emission at 590 nm with a SpectraMax Gemini EM microplate reader. Readings from wells without cells were used as E_{blank}, and the readings from control cells without treatment (E_{control}) represented 100% cell viability. The viability of treated cells was calculated by the following equation:

Cell viability=100×(Eₛₐₘₚₗₑ−E_{blank})/(E_{control}−E_{blank})%

For NIO cells, rPR-OH and rPR-DFO exhibited significantly less cytotoxicity than free DFO, with free DFO inhibiting 50% cell viability at ca. 10 μM and rPR-DFO at ca. >1000 μM (FIG. 8); the cytotoxic behavior of rPR-OH and rPR-DFO compared to DFO was similar on IO cells (FIG. 9).

The elevated presence of iron in cells correlates indirectly with the concentration of ferritin expressed by cells for its storage. J774A.1 mouse macrophage cells were IO with 100 μM FAC for 24 h to increase their overall ferritin expression levels and then treated with rPR-OH, rPR-DFO or DFO for 48 h. A mouse ferritin ELISA assay was used to measure the concentration of ferritin present in cells after treatment. J774A.1 macrophage cells were seeded in 6-well plates at a density of 30,000 cells per well and allowed to settle for 24 h at 37° C., 5% CO2 and 100% humidity with DMEM complete medium. Cells were then treated with 100 μM FAC for 24 h, washed with PBS and treated with free DFO or rPR-DFO at 10 μM or 50 μM equivalent DFO concentrations. After 48 h incubation, cells were lysed with cell lysis buffer (150 mM NaCl, 10 mM Tris, 1% Triton X-100 and protease inhibitor cocktail, pH 7.4). Total protein concentration was measured with the BCA protein assay kit and cellular ferritin concentration was measured with a mouse ferritin ELISA kit. The results are plotted as the ratio of ng of ferritin per μg total protein concentration.

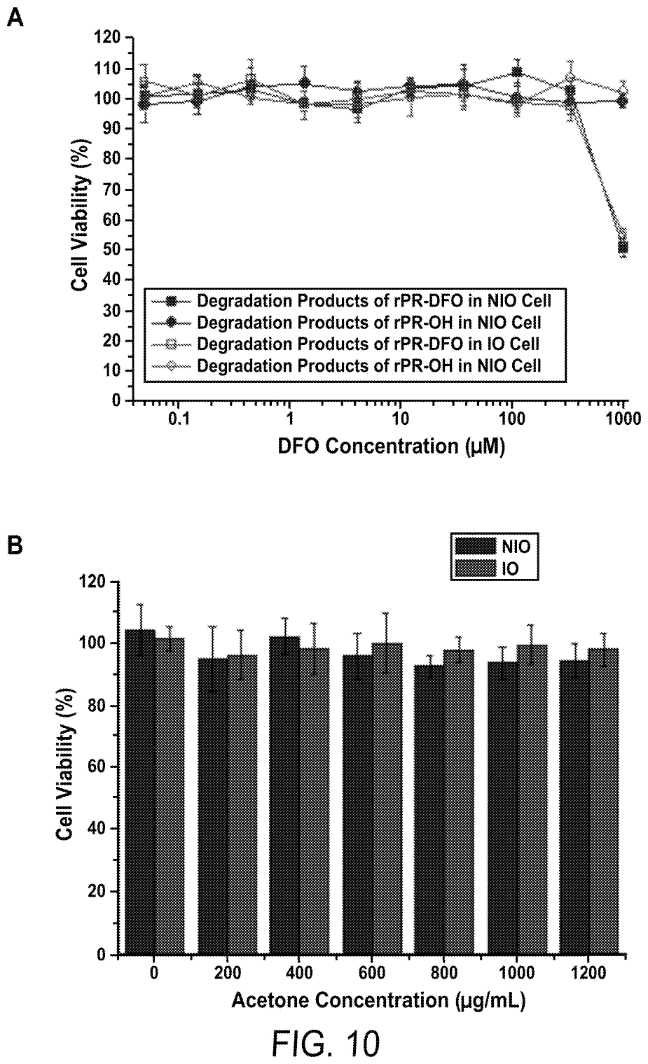

As shown in FIG. 10, 10 μM free DFO and equivalent rPR-DFO were similarly able to reduce ferritin concentrations in cells and this ferritin reduction effect was even more pronounced at 50 μM DFO and rPR-DFO; equivalent concentrations of the carrier rPR-OH had no effect on ferritin concentrations.

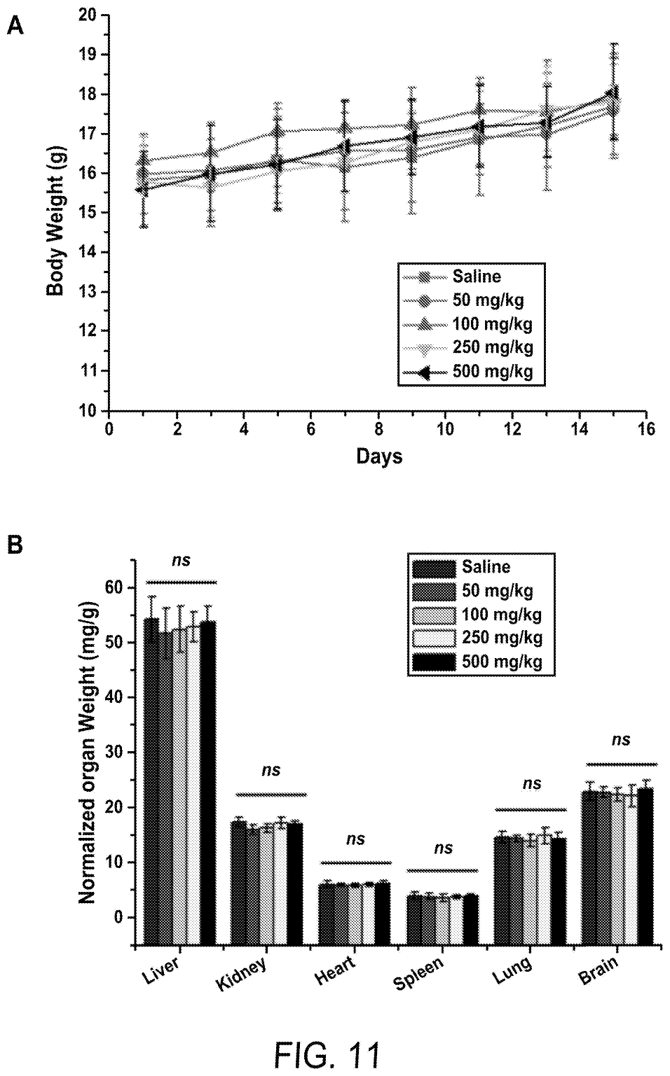

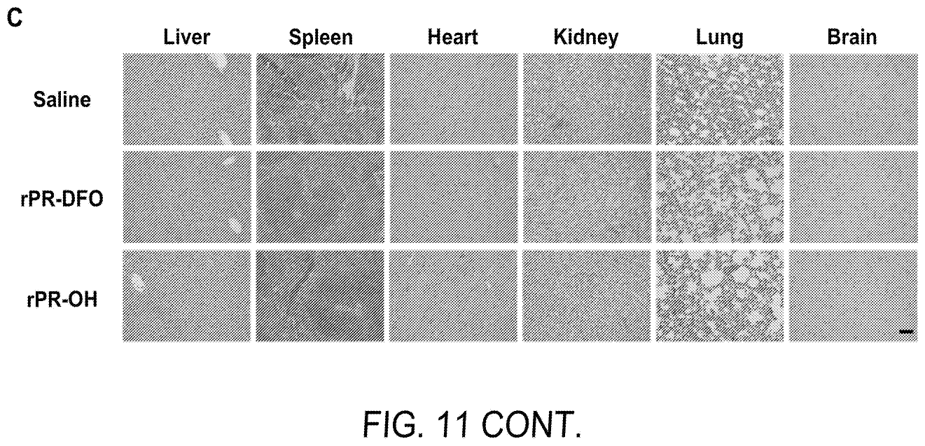

Normal NIO mice (Balb/C, Female, 6 weeks old), randomly divided into 7 groups (n=6), were treated with saline, single-dose (300 mg/kg DFO equiv) or multiple-dose (100 mg/kg DFO equiv every 2 days, six doses total) regimens of DFO, rPR-DFO, and rPR-OH (same concentrations as rPR-DFO based on w/v) via tail-vein injections (10 μL/g). Mice were monitored for 14 days for signs of acute toxicity based on body weight loss, changes in appetite, and changes in behavior such as altered gait and lethargy. The body weights of the mice were recorded every other day over the course of the study. Mice were euthanized by CO₂ overdose on the 14th day. The lungs, heart, spleen, kidneys, brain and liver of animals were subsequently harvested, rinsed with fresh PBS, blotted dry with Whatman filter paper, and then weighed (note that organ weight is reported as mg of total organ weight per g of animal bw, mg/g). There was no apparent evidence of acute toxicity based on normal body weights and organ weights for all treatment groups compared to saline-treated controls (FIG. 11). Mice also behaved normally and no changes in appetite were noted.

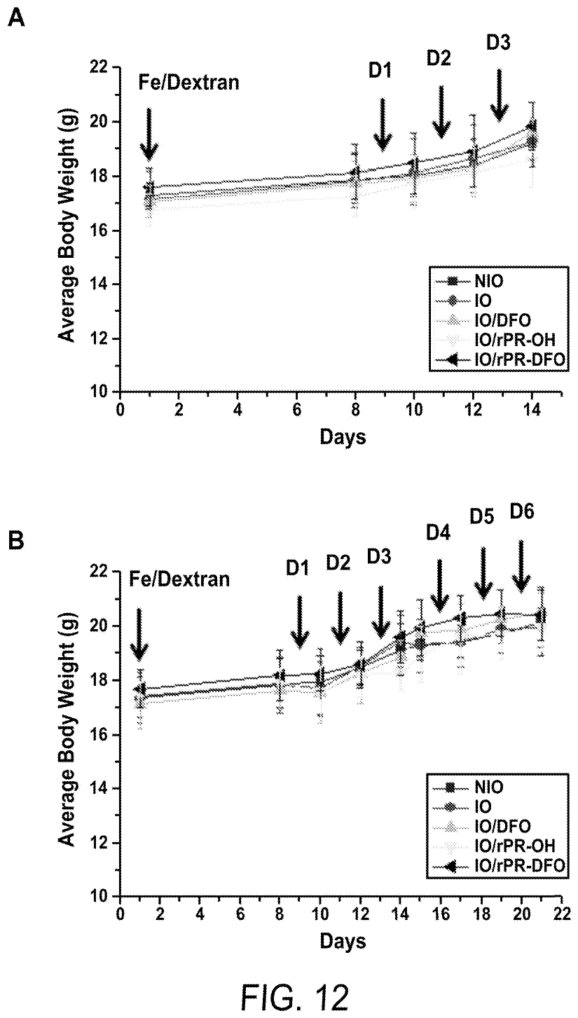

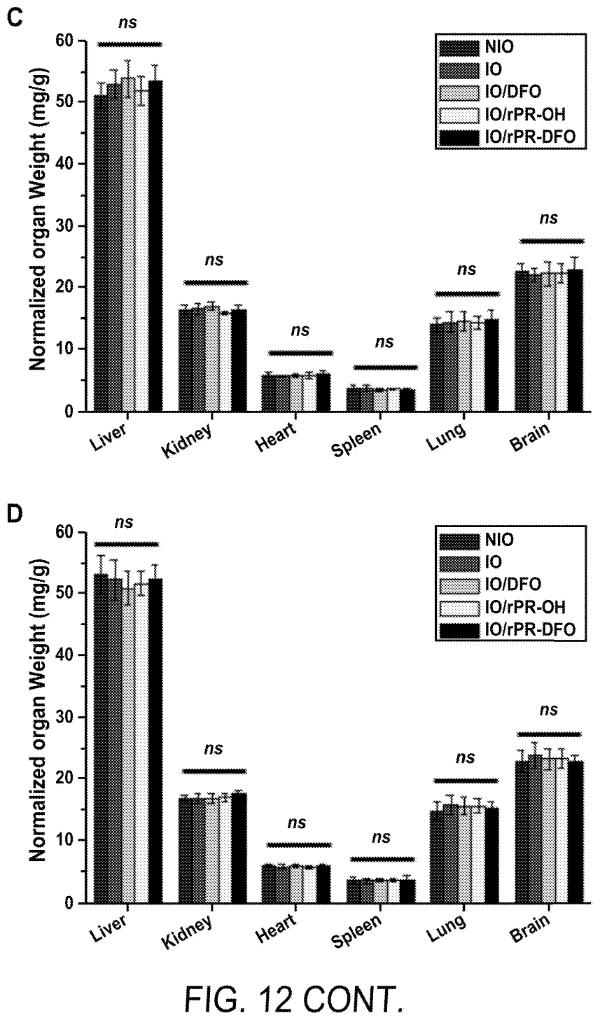

FIG. 12: Efficacy study in an iron overload mouse model

All animal experiments were conducted in accordance with the University of Georgia's IACUC guidelines and the NIH Guide for the Care and Use of Laboratory Animals. Female Balb/C mice, 6 weeks old, were housed in a room maintained at 20±1° C. and with 12 h light and dark cycles. Feed (Harlan Teklad 8604 Rodent Diet) and water were available ad libitum. Mice were IO by a single tail vein injection of Dextran/Fe (Anem-X 100, Aspen Veterinary Resources, Ltd; 50 mg/kg of Fe, 10 μl/g BW in normal saline) on Day 1. The mice were monitored daily for one week to ensure iron overload levels remained constant based on serum ferritin measurements. On Day 8, mice were randomly housed and started on iron-deficient powder diet (Teklad TD.80396.PWD) ad libitum.

Mice were randomly divided into 10 groups (n=3/group) on day 8 and treated with the various formulations (10 μl/g BW) with either 3 or 6 treatments every other day: Group 1-1=normal NIO mice received saline for a total of 3 doses; Group 1-2=normal NIO mice received saline for a total of 6 doses; Group 2-1=IO mice received saline for a total of 3 doses; Group 2-2=IO mice received saline for a total of 6 doses; Group 3-1=IO mice received DFO at 100 mg/kg for a total of 3 doses; Group 3-2=IO mice received DFO at 100 mg/kg for a total of 6 doses; Group 4-1=IO mice received rPR-DFO at an equivalent concentration of 100 mg/kg DFO for a total of 3 doses; Group 4-2=IO mice received rPR-DFO at an equivalent concentration of 100 mg/kg DFO for a total of 6 doses. Group 5-1=IO mice received equivalent rPR-OH based on w/v to rPR-DFO concentrations for a total of 3 doses; Group 5-2=IO mice received equivalent rPR-OH based on w/v to rPR-DFO concentrations for a total of 6 doses. At the end of the treatment period of either 3 or 6 doses, mice were sacrificed and blood collected by cardiac puncture to measure final serum ferritin levels by ELISA assay (FIG. 12).

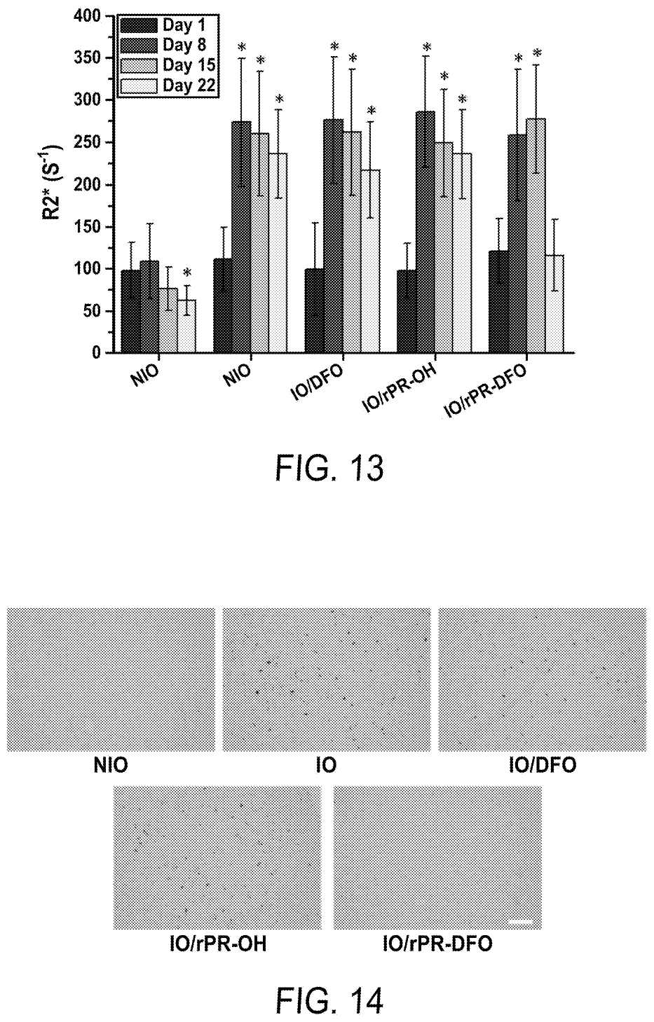

The lungs, heart, spleen, kidneys, brain and liver of animals were subsequently harvested, rinsed with fresh PBS, blotted dry with Whatman filter paper, and then weighed (note that organ weight is reported as mg of total organ weight per g of animal bw, mg/g). There was no apparent evidence of acute toxicity based on normal body weights and organ weights for all groups treated with 3 or 6 doses compared to saline-treated controls (FIG. 13). Mice also behaved normally and no changes in appetite were noted.

IO mice were treated with 6 doses of rPR-DFO or DFO every 2 days as described above. Magnetic resonance imaging using R2* measurements reveal a significant decrease in liver iron concentrations back to normal range only in mice treated with rPR-DFO by Day 21 in contrast to untreated IO mice or DFO-treated rodents (FIG. 14).

SulfoCy5.5-labeled rPR-DFO (50 mg/kg, 10 μl/g BW in normal saline) was injected to IO or NIO mice via tail vein. In vivo real-time NIRF images were acquired on a Maestro II imaging system (PerkinElmer) using a red emission filter (670-900 nm). The mice were anesthetized with isoflurane at different time points post injection for imaging up to 192 h. (FIG. 18). At the end of the study, the mouse was euthanized and heart, liver, spleen, kidneys, lung and blood were harvested for ex vivo imaging (FIG. 19). Total urine and feces were also collected for imaging (FIG. 20).

Confocal laser scanning microscopy (CLSM) was used to track the cellular uptake behavior of rPR-DFO after labeling with fluorescein cadaverine (Fc). Specifically, cells were seeded in glass-bottom cell culture dishes (NEST Biotechnology Co., LTD) at a density of 10,000 cells/well and allowed to settle for 24 h at 37° C. The next day, cells were incubated at 37° C., 5% CO₂ in DMEM culture medium (with or without 100 μM FAC) for another 24 h and subsequently washed with PBS prior to returning to the incubator for an additional 48 h. Next, cells were washed 3× with PBS at RT and incubated with 5 mg/mL Fc-labeled rPR-DFO in culture medium for 4 h and then LysoTracker® Red DND-99 in culture medium at a final concentration of 1 μg/mL for 30 min at 37° C. Afterwards, cells were washed 3× with PBS and fixed with 4% paraformaldehyde (PFA) for 10 min at room temperature prior to imaging. The cellular association and intracellular localization of Fc-labeled rPR-DFO were observed with a Zeiss LSM 710 Confocal Microscope. All fluorescence images were captured via green and red channels. Typical CLSM image shows Fc-labeled rPR-DFO is internalized by both NIO and IO cells. Cell morphology appeared normal and rPR-DFO was predominantly observed to co-localize with LysoTracker in endolysosomes of NIO and IO cells.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A polyrotaxane comprising:
a) A plurality of macrocycles, wherein each macrocycle has an inner cavity, wherein at least one macrocycle is conjugated to a metal chelating group;
b) a skewer molecule comprising a linear portion disposed through the inner cavity of each macrocycle and a blocking group disposed at each end of the linear portion, wherein at least one blocking group is selectively cleavable from the linear portion.

2. The polyrotaxane according to claim 1, wherein the macrocycles comprise cyclic polysaccharides, cyclic polypeptides, crown ethers, cucurbiturals, calixarenes, and pillararenes, or combinations thereof.

3. The polyrotaxane according to claim 1, wherein the macrocycles comprise cyclodextrins.

4. The polyrotaxane according to claim 1, wherein the macrocycles comprise α-cyclodextrin, β-cyclodextrin, γ-cyclodextrin, or a combination thereof.

5. The polyrotaxane according to claim 1, wherein the metal chelating group comprises an iron chelator.

6. The polyrotaxane according to claim 1, wherein the metal chelating group comprises deferoxamine, desferasirox, deferiprone, or a combination thereof.

7. The polyrotaxane according to claim 1, wherein the linear portion of the skewer molecule comprises a polyethylene glycol, polyester, polycarbonate, polyvinyl alcohol, polyanhydride, polyacetal, or a copolymer thereof.

8. (canceled)

9. The polyrotaxane according to claim 1, wherein the skewer molecule comprises a blocking group covalently bonded to the linear portion via at least one of a thioacetal group, an acetal group, an oxalate, a bis(cyclopentadienyl) metal complex, an imine group, a selenide bond, a boronic ester, or a combination thereof.

10. The polyrotaxane according to claim 9, wherein the blocking group comprises an adamantyl group, a triphenylmethyl group, a cyclodextrin, amino acid, pyrene, or a combination thereof.

11. The polyrotaxane according to claim 1, wherein at least one macrocycle is conjugating to a solubilizing group.

12. The polyrotaxane according to claim 11, wherein the solubilizing group comprises an C₁₋₄ alkyl alcohol, C₁₋₄ alkyl amine, C₁₋₄ alkyl carboxylate, C₁₋₄ alkyl phosphonate, C₁₋₄ alkyl sulfonate,

13. A polyrotaxane having the formula: 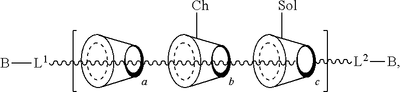 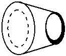
represents an unfunctionalized cyclodextrin, and a is selected from 0-50; 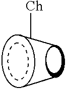
represents a cyclodextrin conjugated with at least one metal chelator, and b is selected from 1-50; 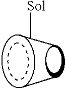
represents a cyclodextrin conjugated with at least one solubilizing group, and c is selected from 0-50;
wherein [symbol] represents a linear polymer disposed through the inner cavity of each cyclodextrin,
L¹ and L² are each linkers, provided at least one of L¹ or L² is a cleavable linker, and
B is a blocking group.

14. The polyrotaxane according to claim 13, wherein the metal chelating group comprises an iron chelator.

15. The polyrotaxane according to claim 13, wherein the metal chelating group comprises deferoxamine, desferasirox, deferiprone, or a combination thereof.

16. The polyrotaxane according to claim 13, wherein 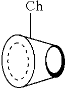
represents a macrocycle having the formula: 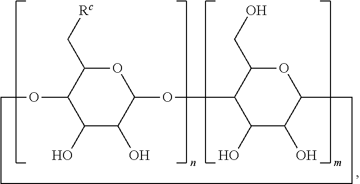
wherein n is selected from 1-8, m is selected from 0-7, wherein the sum of n+m is from 6-8;
and R^{c} is—is selected from —O—X¹—Z, —NH—X¹—Z, —NHC(═O)—X¹—Z, OC(═O)NH—X¹—Z, wherein X¹ is absent, aryl, alkaryl, or (CH₂)ₓ, wherein x is selected from 1-10 and Z is a metal chelator.

17. (canceled)

18. The polyrotaxane according to claim 16, wherein R^{c} has the structure:

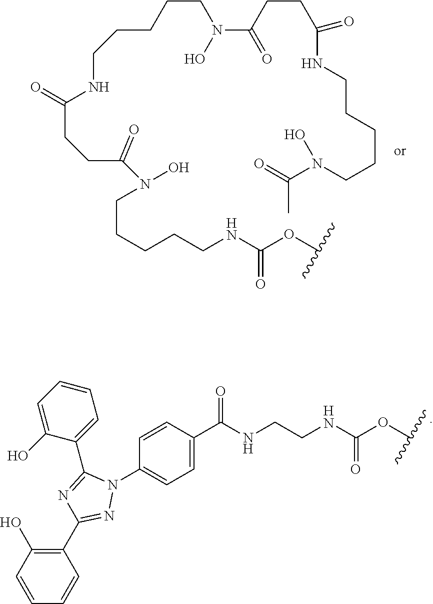

19. The polyrotaxane according to claim 13, wherein 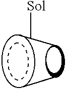
represents a macrocycle having the formula: 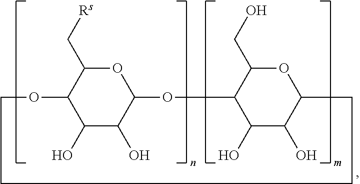
and R^{s} is—is selected from —O—(CH₂)_{y}—Y, —NH—(CH₂)_{y}—Y, —NHC(═O)—(CH₂)_{y}—Y, OC(═O)NH—(CH₂)_{y}—Y, wherein X¹ is (CH₂)_{y}, wherein y is selected from 1-4 and Z is selected from OH, NH₂, COOH, SO₃H, PO₃H₂, and pharmaceutically acceptable salts thereof.

20. (canceled)

21. The polyrotaxane according claim 13, wherein B has the formula: 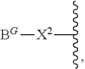
wherein X² is absent, —NH—, —O—, —C(═O)NH—, —NHC(═O)NH—, —NHC(═O)O—, —OC(═O)NH—, —NHC(═O)—; and
B^{G} is adamantyl, triphenylmethyl, or a cyclodextrin.

22. The polyrotaxane according to claim 13, wherein L¹ and L² are independently selected from: 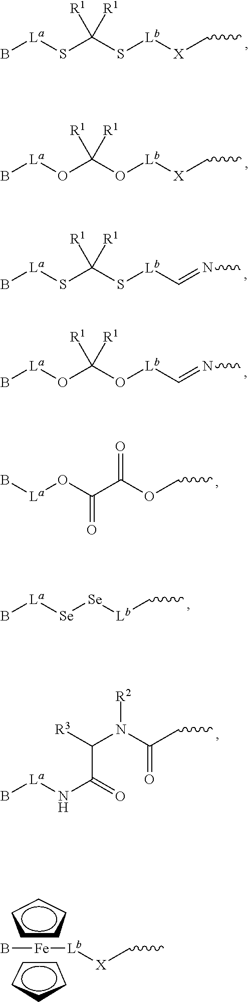
wherein X is O, NH, S, or absent;
R¹, R² and R³ are in each case independently selected from H, C₁₋₈ alkyl, aryl, and any two of R¹, R², and R³ may together form a ring;
L^{a} is —(CH₂)_{z}, wherein z is 0-10; and
L^{b} is —(CH₂)_{y}, wherein y is 1-10.

23. A method of treating iron overload in a patient comprising administering to the patient a composition comprising the polyrotaxane according to claim 1.
